(19) United States
(12) Patent Application Publication
(10) Pub. No.: US 2020/0403163 A1
(43) Pub. Date: Dec. 24, 2020

(54) PHOTOELECTRIC CONVERSION ELEMENT, IMAGING ELEMENT, OPTICAL SENSOR, AND COMPOUND

(71) Applicant: FUJIFILM Corporation, Tokyo (JP)

(72) Inventors: Tomoaki YOSHIOKA, Kanagawa (JP); Eiji FUKUZAKI, Kanagawa (JP); Tomoyuki MASHIKO, Kanagawa (JP)

(73) Assignee: FUJIFILM Corporation, Tokyo (JP)

(21) Appl. No.: 17/010,811

(22) Filed: Sep. 2, 2020

Related U.S. Application Data
(63) Continuation of application No. PCT/JP2019/009234, filed on Mar. 8, 2019.

(30) Foreign Application Priority Data
Mar. 12, 2018 (JP) 2018-044292

(51) Int. Cl. H01L 51/00; C07D 219/02; C07D 409/04; C07D 495/10; C07D 401/06

(52) U.S. Cl. CPC H01L 51/0072; C07D 219/02; H01L 51/0053; C07D 409/04; H01L 27/307; C07D 495/10; H01L 51/0071; C07D 401/06; H01L 51/0067; H01L 51/0068

(57) ABSTRACT

The present invention provides a photoelectric conversion element having a photoelectric conversion film having a narrow half-width of absorption peak, an imaging element, an optical sensor, and a compound. The photoelectric conversion element according to the embodiment of the present invention includes a conductive film, a photoelectric conversion film, and a transparent conductive film, in this order, in which the photoelectric conversion film contains a compound represented by Formula (1).

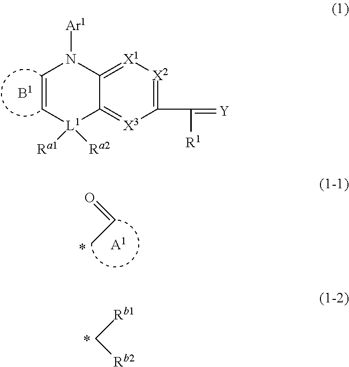

### CROSS-REFERENCE TO RELATED APPLICATIONS

This application is a Continuation of PCT International Application No. PCT/JP2019/009234 filed on Mar. 8, 2019, which claims priority under 35 U.S.C. § 119(a) to Japanese Patent Application No. 2018-044292 filed on Mar. 12, 2018. The above application is hereby expressly incorporated by reference, in its entirety, into the present application.

### BACKGROUND OF THE INVENTION

### 1. Field of the Invention

The present invention relates to a photoelectric conversion element, an imaging element, an optical sensor, and a compound.

### 2. Description of the Related Art

In recent years, development of an element having a photoelectric conversion film (for example, an imaging element) has progressed.

Regarding a photoelectric conversion element using a photoelectric conversion film, for example, JP2012-077064A discloses a photoelectric conversion element having a photoelectric conversion film containing a predetermined compound.

### SUMMARY OF THE INVENTION

As one aspect of an imaging element, there is a laminated type imaging element in which a plurality of photoelectric conversion elements that receive different types of light are laminated. In a case where light is incident on the imaging element, a part of the incidence ray is absorbed by the photoelectric conversion elements arranged on the incident side, and the transmitted light is absorbed by the photoelectric conversion elements arranged further inside. In such an imaging element, since colors are easily separated, it is preferable that the absorption peak of each photoelectric conversion element has a narrow half-width.

The present inventors have examined the characteristics of the photoelectric conversion element disclosed in JP2012-077064A, and have found that the half-width of the absorption peak of the photoelectric conversion film in the photoelectric conversion element is wide, and further improvement is necessary.

In view of the circumstances, an object of the present invention is to provide a photoelectric conversion element having a photoelectric conversion film having a narrow half-width of absorption peak.

Another object of the present invention is to provide an imaging element, an optical sensor, and a compound.

The inventors of the present invention have conducted extensive studies on the above-described problems. As a result, the inventors have found that it is possible to solve the above-described problems by applying the compound having a predetermined structure to the photoelectric conversion film, and have completed the present invention.

(1) A photoelectric conversion element comprising a conductive film, a photoelectric conversion film, and a transparent conductive film, in this order, in which the photoelectric conversion film contains a compound represented by Formula (1) described below.

(2) The photoelectric conversion element according to (1), in which the compound represented by Formula (1) described below is a compound represented by Formula (2) described below.

(3) The photoelectric conversion element according to (1) or (2), in which the compound represented by Formula (1) described below is a compound represented by Formula (3) described below.

(4) The photoelectric conversion element according to any one of (1) to (3), in which the compound represented by Formula (1) described below is a compound represented by Formula (4) described below.

(5) The photoelectric conversion element according to any one of (1) to (4), in which R^{a1} and R^{a2} each independently represent a substituent having 3 or more carbon atoms.

(6) The photoelectric conversion element according to any one of (1) to (5), in which R^{a1} and R^{a2} each independently represent a secondary alkyl group having 3 or more carbon atoms, an aryl group having 3 or more carbon atoms, or a heteroaryl group having 3 or more carbon atoms.

(7) The photoelectric conversion element according to any one of (1) to (6), in which the photoelectric conversion film further contains an n-type organic semiconductor, and has a bulk hetero structure formed in a state where the compound represented by Formula (1) and the n-type organic semiconductor are mixed.

(8) The photoelectric conversion element according to any one of (1) to (7), further comprising one or more interlayers between the conductive film and the transparent conductive film, in addition to the photoelectric conversion film.

(9) An imaging element comprising the photoelectric conversion element according to any one of (1) to (8).

(10) The imaging element according to (9), further comprising another photoelectric conversion element that receives light having a wavelength different from light received by the photoelectric conversion element.

(11) The imaging element according to (10), in which the photoelectric conversion element and the other photoelectric conversion element are laminated, and at least a part of incidence ray is transmitted through the photoelectric conversion element, and then is received by the other photoelectric conversion element.

(12) The imaging element according to (10) or (11), in which the photoelectric conversion element is a green photoelectric conversion element, and the other photoelectric conversion element includes a blue photoelectric conversion element and a red photoelectric conversion element.

(13) An optical sensor comprising the photoelectric conversion element according to any one of (1) to (8).

(14) A compound represented by Formula (1).

(15) The compound according to (14) which is represented by Formula (2).

(16) The compound according to (14) or (15) which is represented by Formula (3).

(17) The compound according to any one of (14) to (16) which is represented by Formula (4).

(18) The compound according to any one of (14) to (17), in which R^{a1} and R^{a2} each independently represent a substituent having 3 or more carbon atoms.

(19) The compound according to any one of (14) to (18), in which R^{a1} and R^{a2} each independently represent a secondary alkyl group having 3 or more carbon atoms, an aryl group having 3 or more carbon atoms, or a heteroaryl group having 3 or more carbon atoms.

According to the present invention, it is possible to provide a photoelectric conversion element having a photoelectric conversion film having a narrow half-width of absorption peak. According to the present invention, it is possible to provide an imaging element, an optical sensor, and a compound.

### BRIEF DESCRIPTION OF THE DRAWINGS

FIG. 1 is a schematic cross-sectional view showing an example of a configuration of a photoelectric conversion element.

FIG. 2 is a schematic cross-sectional view showing an example of a configuration of a photoelectric conversion element.

FIG. 3 is a schematic cross-sectional view of one embodiment of an imaging element.

FIG. 4 is a schematic cross-sectional view of another embodiment of an imaging element.

FIG. 5 is a ¹H nuclear magnetic resonance (NMR) spectrum of a compound (D-1).

FIG. 6 is a ¹H NMR spectrum of a compound (D-2).

FIG. 7 is a ¹H NMR spectrum of a compound (D-6).

FIG. 8 is a ¹H NMR spectrum of a compound (D-7).

FIG. 9 is a ¹H NMR spectrum of a compound (D-8).

FIG. 10 is a ¹H NMR spectrum of a compound (D-9).

FIG. 11 is a ¹H NMR spectrum of a compound (D-10).

FIG. 12 is a ¹H NMR spectrum of a compound (D-11).

### DESCRIPTION OF THE PREFERRED EMBODIMENTS

Hereinafter, preferred embodiments of a photoelectric conversion element of the present invention will be described.

In the present specification, a substituent for which whether it is substituted or unsubstituted is not specified may be further substituted with a substituent (for example, a substituent W described below) within the scope not impairing an intended effect. For example, the expression of “alkyl group” refers to an alkyl group with which a substituent (for example, a substituent W described below) may be substituted.

In addition, in the present specification, the numerical range represented by “to” means a range including numerical values denoted before and after “to” as a lower limit value and an upper limit value.

There is a feature of the photoelectric conversion element according to the embodiment of the present invention is that a bulky substituent is introduced into a compound represented by Formula (1) described below (hereinafter, also referred to as “specific compound”) contained in the photoelectric conversion film. More specifically, by introducing a bulky substituent at the positions of R^{a1} and R^{a2} in Formula (1), the structure of the specific compound itself is twisted and the association of the specific compounds in the photoelectric conversion film is suppressed, as a result, it is presumed that the half-width of the absorption peak of the photoelectric conversion film is narrowed.

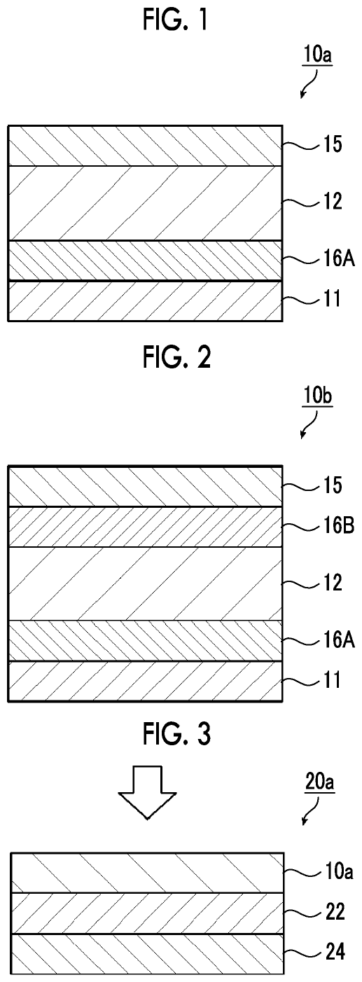

FIG. 1 shows a schematic cross-sectional view of one embodiment of a photoelectric conversion element of the present invention.

A photoelectric conversion element 10a shown in FIG. 1 has a configuration in which a conductive film (hereinafter, also referred to as a lower electrode) 11 functioning as the lower electrode, an electron blocking film 16A, a photoelectric conversion film 12 containing the specific compound described below, and a transparent conductive film (hereinafter, also referred to as an upper electrode) 15 functioning as the upper electrode are laminated in this order.

FIG. 2 shows a configuration example of another photoelectric conversion element. A photoelectric conversion element 10b shown in FIG. 2 has a configuration in which the electron blocking film 16A, the photoelectric conversion film 12, a positive hole blocking film 16B, and the upper electrode 15 are laminated on the lower electrode 11 in this order. The lamination order of the electron blocking film 16A, the photoelectric conversion film 12, and the positive hole blocking film 16B in FIGS. 1 and 2 may be appropriately changed according to the application and the characteristics.

In the photoelectric conversion element 10a (or 10b), it is preferable that light is incident on the photoelectric conversion film 12 through the upper electrode 15.

In a case where the photoelectric conversion element 10a (or 10b) is used, the voltage can be applied. In this case, it is preferable that the lower electrode 11 and the upper electrode 15 form a pair of electrodes and the voltage of 1×10⁻⁵ to 1×10⁷ V/cm is applied thereto. From the viewpoint of performance and power consumption, the voltage to be applied is more preferably 1×10⁻⁴ to 1×10⁷ V/cm, and still more preferably 1×10⁻³ to 5×10⁶ V/cm. The voltage application method is preferable that the voltage is applied such that the electron blocking film 16A side is a cathode and the photoelectric conversion film 12 side is an anode, in FIGS. 1 and 2. In a case where the photoelectric conversion element 10a (or 10b) is used as an optical sensor, or also in a case where the photoelectric conversion element 10a (or 10b) is incorporated in an imaging element, the voltage can be applied by the same method.

As described in detail below, the photoelectric conversion element 10a (or 10b) can be suitably applied to applications of the imaging element.

Hereinafter, the form of each layer constituting the photoelectric conversion element according to the embodiment of the present invention will be described in detail.

<Photoelectric Conversion Film>

The photoelectric conversion film is a film containing a specific compound as a photoelectric conversion material. By using the compound, a photoelectric conversion element having a photoelectric conversion film having a narrow half-width of absorption peak can be obtained.

Hereinafter, the specific compound will be described in detail.

Formula (1) includes all geometric isomers that can be distinguished based on the C═C double bond constituted by a carbon atom to which R bonds and a carbon atom adjacent thereto in Formula (1). That is, both the cis isomer and the trans isomer which are distinguished based on the C═C double bond are included in Formula (1).

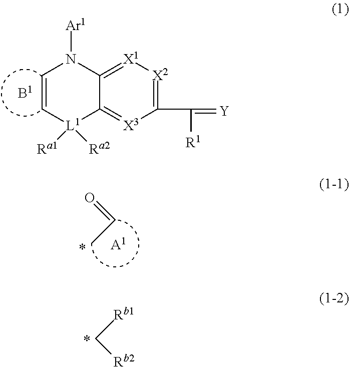

In Formula (1), Ar¹ represents an aryl group which may have a substituent or a heteroaryl group which may have a substituent.

The carbon atoms of the aryl group are not particularly limited, but is preferably 6 to 30, more preferably 6 to 18, and still more preferably 6. The aryl group may have a monocyclic structure or a condensed ring structure (a fused ring structure) in which two or more rings are condensed.

As the aryl group, for example, a phenyl group, a naphthyl group, or an anthryl group is preferable, and a phenyl group is more preferable.

Examples of the substituent that the aryl group may have include the substituent W described below, and include an alkyl group.

The aryl group may have a plurality of types of substituents.

In a case where the aryl group has a substituent, the number of substituents that the aryl group has is not particularly limited, but is preferably 1 to 5, and more preferably 2 to 3, from a point that the half-width of the absorption peak of the photoelectric conversion film becomes narrower (hereinafter, simply referred to as “the viewpoint of obtaining a superior effect of the present invention”).

The carbon atoms of the heteroaryl group (a monovalent aromatic heterocyclic group) are not particularly limited, but is preferably 3 to 30, and more preferably 3 to 18.

The heteroaryl group includes a hetero atom in addition to a carbon atom and a hydrogen atom. Examples of the hetero atom include a sulfur atom, an oxygen atom, a nitrogen atom, a selenium atom, a tellurium atom, a phosphorus atom, a silicon atom, and a boron atom, and a sulfur atom, an oxygen atom, or a nitrogen atom is preferable.

The number of hetero atoms of the heteroaryl group is not particularly limited, but is preferably 1 to 10, more preferably 1 to 4, and still more preferably 1 to 2.

The number of ring members of the heteroaryl group is not particularly limited, but is preferably 3 to 8, more preferably 5 to 7, and still more preferably 5 to 6. The heteroaryl group may have a monocyclic structure or a condensed ring structure in which two or more rings are condensed. In a case of the condensed ring structure, an aromatic hydrocarbon ring having no hetero atom (for example, a benzene ring) may be included.

Examples of the heteroaryl group include a furyl group, a pyridyl group, a quinolyl group, an isoquinolyl group, an acridinyl group, a phenanthridinyl group, a pteridinyl group, a pyrazinyl group, a quinoxalinyl group, a pyrimidinyl group, a quinazolyl group, a pyridazinyl group, a cinnolinyl group, a phthalazinyl group, a triazinyl group, an oxazolyl group, a benzoxazolyl group, a thiazolyl group, a benzothiazolyl group, an imidazolyl group, a benzimidazolyl group, a pyrazolyl group, an indazolyl group, an isoxazolyl group, a benzisoxazolyl group, an isothiazolyl group, a benzisothiazolyl group, an oxadiazolyl group, a thiadiazolyl group, a triazolyl group, a tetrazolyl group, a benzofuryl group, a thienyl group, a benzothienyl group, a dibenzofuryl group, a dibenzothienyl group, a pyrrolyl group, an indolyl group, an imidazopyridinyl group, and a carbazolyl group.

Among these, a furyl group, a thienyl group, a pyridyl group, a quinolyl group, an isoquinolyl group, or a carbazolyl group is preferable.

Examples of the substituent that the heteroaryl group may have include the same substituent that the aryl group may have.

In a case where the heteroaryl group has a substituent, the number of substituents that the heteroaryl group has is not particularly limited, but is preferably 1 to 5, and more preferably 1 to 3.

R¹ represents a hydrogen atom or a substituent. Among these, from the viewpoint of obtaining a superior effect of the present invention, R¹ is preferably a hydrogen atom.

The definition of the above-described substituent is synonymous with the substituent W described below. Examples of the substituent include an alkyl group, an aryl group, and a heteroaryl group.

X¹ to X³ each independently represent CR²(═CR²—) or a nitrogen atom (═N—). R² represents a hydrogen atom or a substituent. The definition of the substituent is synonymous with the substituent W described below. Among these, examples of the substituent include an alkyl group, an aryl group, and a heteroaryl group.

R^{a1} and R^{a2} each independently represent a substituent having 2 or more carbon atoms. The number of carbon atoms contained in the substituent having 2 or more carbon atoms is preferably 3 or more from the viewpoint of obtaining a superior effect of the present invention. That is, the substituent having 2 or more carbon atoms is preferably a substituent having 3 or more carbon atoms. The upper limit of the carbon atoms is not particularly limited, but is 10 or less, for example.

Examples of the substituent having 2 or more carbon atoms include an aliphatic hydrocarbon group having 2 or more carbon atoms, which may contain a hetero atom, and an aromatic group having 2 or more carbon atoms.

Examples of the aliphatic hydrocarbon group include an alkyl group, an alkenyl group, and an alkynyl group. These groups may contain a hetero atom such as an oxygen atom, a nitrogen atom, and a sulfur atom.

Examples of the aromatic groups include an aryl group and a heteroaryl group.

Among these, from the viewpoint of obtaining a superior effect of the present invention, the substituent having 2 or more carbon atoms is preferably a secondary alkyl group having 3 or more carbon atoms, an aryl group having 3 or more carbon atoms, or a heteroaryl group having 3 or more carbon atoms.

The secondary alkyl group means an alkyl group having a secondary carbon atom.

Examples of the secondary alkyl group having 3 or more carbon atoms include an isopropyl group, an isobutyl group, a pentan-2-yl group, a pentan-3-yl group, and a 3-methyl-2-pentylgroup.

Examples of the aryl group having 3 or more carbon atoms include the groups exemplified as the aryl group represented by Ar¹.

Examples of the heteroaryl group having 3 or more carbon atoms include the groups exemplified as the heteroaryl group represented by Ar¹.

L¹ represents a carbon atom, a silicon atom, or a germanium atom. Among these, from the viewpoint of obtaining a superior effect of the present invention, a carbon atom is preferable.

B¹ represents an aromatic ring which may have a substituent.

The aromatic ring may be monocyclic or polycyclic.

Examples of the aromatic ring include an aromatic hydrocarbon ring and an aromatic heterocyclic ring. Examples of the aromatic hydrocarbon ring include a benzene ring, a naphthalene ring, an anthracene ring, and a phenanthrene ring. Examples of the aromatic heterocyclic ring include a pyrrole ring, a furan ring, a thiophene ring, an imidazole ring, and an oxazole ring.

Among these, from the viewpoint of obtaining a superior effect of the present invention, the aromatic hydrocarbon ring is preferable, and a benzene ring is more preferable.

The definition of the substituent is synonymous with the substituent W described below. Examples of the substituent include an alkyl group, an aryl group, and a heteroaryl group. These groups may further have a substituent.

Y represents a group represented by Formula (1-1) or a group represented by Formula (1-2). Among these, from the viewpoint of obtaining a superior effect of the present invention, a group represented by Formula (1-1) is preferable. * in Formulae (1-1) and (1-2) represents a bonding position.

A¹ represents a ring containing at least two carbon atoms. The two carbon atoms refer to a carbon atom in a carbonyl group specified in Formula (1-1) and a carbon atom specified in Formula (1-1), which is adjacent to the carbon atom in a carbonyl group, and both the carbon atoms are atoms constituting A¹.

Further, in the ring, carbon atoms constituting the ring may be substituted with another carbonyl carbon (>C═O) or thiocarbonyl carbon (>C═S). The term “another carbonyl carbon (>C═O)” as used herein means a carbonyl carbon having a carbon atom constituting a ring other than the carbonyl carbon specified in Formula (1-1).

Among these, the carbon atoms of A¹ are preferably 3 to 30, more preferably 3 to 20, and still more preferably 3 to 15. The above-described carbon atoms are a number containing two carbon atoms specified in Formula.

A¹ may have a hetero atom, and for example, is preferably a nitrogen atom, a sulfur atom, an oxygen atom, a selenium atom, a tellurium atom, a phosphorus atom, a silicon atom, and a boron atom, and a nitrogen atom, a sulfur atom, or an oxygen atom, and more preferably an oxygen atom.

The number of hetero atoms in A¹ is preferably 0 to 10, more preferably 0 to 5, and still more preferably 0 to 2. The number of the hetero atoms is the number excluding the number of hetero atoms (which are intended to include the carbonyl carbon specified in Formula (1-1)) introduced into the ring by replacing the carbon atom constituting the ring represented by A¹ with the carbonyl carbon (>C═O) or the thiocarbonyl carbon (>C═S), and the number of hetero atoms that the substituent of A¹ has.

A¹ may have a substituent, and the substituent is preferably a halogen atom (preferably chlorine atom), an alkyl group (which may be any of linear, branched, or cyclic, and preferably has 1 to 10 carbon atoms, more preferably 1 to 6 carbon atoms), an aryl group (which preferably has 6 to 18 carbon atoms, more preferably 6 carbon atoms), a heteroaryl group (the carbon numbers are preferably 5 to 18, and more preferably 5 to 6), or a silyl group (which may be linear, branched, or cyclic, and the number of silicon is preferably 1 to 4, and more preferably 1).

A¹ may or may not indicate aromaticity.

A¹ may have a monocyclic structure or a condensed ring structure, but is preferably a 5-membered ring, a 6-membered ring, or a fused ring containing at least any one of a 5-membered ring or a 6-membered ring. The number of rings forming the fused ring is preferably 1 to 4, and more preferably 1 to 3.

As the ring represented by A¹, a ring which is usually used as an acidic nucleus (specifically, an acidic nucleus in a merocyanine dye) is preferable, and specific examples thereof are as follows.

(a) 1,3-Dicarbonyl nucleus: for example, 1,3-indandione nucleus, 1,3-cyclohexanedione, 5,5-dimethyl-1,3-cyclohexanedione, 1,3-dioxane-4,6-dione, and the like.

(b) Pyrazolinone nucleus: for example, 1-phenyl-2-pyrazolin-5-one, 3-methyl-1-phenyl-2-pyrazolin-5-one, 1-(2-benzothiazolyl)-3-methyl-2-pyrazolin-5-one, and the like.

(c) Isoxazolinone nucleus: for example, 3-phenyl-2-isoxazolin-5-one, 3-methyl-2-isoxazolin-5-one, and the like.

(d) Oxindole nucleus: for example, 1-alkyl-2,3-dihydro-2-oxindole, and the like.

(e) 2,4,6-Trioxohexahydropyrimidine nucleus: for example, barbituric acid, 2-thiobarbituric acid and derivatives thereof, or the like. Examples of the derivative include a 1-alkyl form such as 1-methyl and 1-ethyl, a 1,3-dialkyl form such as 1,3-dimethyl, 1,3-diethyl, and 1,3-dibutyl, a 1,3-diaryl form such as 1,3-diphenyl, 1,3-di(p-chlorophenyl), 1,3-di(p-ethoxycarbonylphenyl), 1-alkyl-1-aryl form such as 1-ethyl-3-phenyl, and a 1,3-diheteroaryl form such as 1,3-di(2-pyridyl).

(f) 2-Thio-2,4-thiazolidinedione nucleus: for example, rhodanine and derivatives thereof. Examples of the derivatives include 3-alkylrhodanine such as 3-methylrhodanine, 3-ethylrhodanine, and 3-allylrhodanine, 3-arylrhodanine such as 3-phenylrhodanine, and 3-heteroaryl rhodanine such as 3-(2-pyridyl)rhodanine.

(g) 2-Thio-2,4-oxazolidinedione nucleus(2-thio-2,4-(3H, 5H)-oxazoledione nucleus: for example, 3-ethyl-2-thio-2,4-oxazolidinedione, and the like.

(h) Tianaphthenone nucleus: for example, 3(2H)-thianaphthenone-1,1-dioxide, and the like.

(i) 2-Thio-2,5-thiazolidinedione nucleus: for example, 3-ethyl-2-thio-2,5-thiazolidinedione, and the like.

(j) 2,4-Thiazolidinedione nucleus: for example, 2,4-thiazolidinedione, 3-ethyl-2,4-thiazolidinedione, 3-phenyl-2,4-thiazolidinedione, and the like.

(k) Thiazolin-4-one nucleus: for example, 4-thiazolinone, 2-ethyl-4-thiazolinone, and the like.

(l) 2,4-Imidazolidinedione (hydantoin) nucleus: for example, 2,4-imidazolidinedione, 3-ethyl-2,4-imidazolidinedione, and the like.

(m) 2-Thio-2,4-imidazolidinedione (2-thiohydantoin) nucleus: for example, 2-thio-2,4-imidazolidinedione, 3-ethyl-2-thio-2,4-imidazolidinedione, and the like.

(n) Imidazolin-5-one nucleus: for example, 2-propylmercapto-2-imidazolin-5-one, and the like.

(o) 3,5-Pyrazolidinedione nucleus: for example, 1,2-diphenyl-3,5-pyrazolidinedione, 1,2-dimethyl-3,5-pyrazolidinedione, and the like.

(p) Benzothiophene-3(2H)-one nucleus: for example, benzothiophene-3(2H)-one, oxobenzothiophene-3(2H)-one, dioxobenzothiophene-3(2H)-one, and the like.

(q) Indanone nucleus: for example, 1-indanone, 3-phenyl-1-indanone, 3-methyl-1-indanone, 3,3-diphenyl-1-indanone, 3,3-dimethyl-1-indanone, and the like.

(r) Benzofuran-3-(2H)-one nucleus: for example, benzofuran-3-(2H)-one, and the like.

(s) 2,2-Dihydrophenalene-1,3-dione nucleus, and the like.

R^{b1} and R^{b2} each independently represent a cyano group or —COOR^{d1}.

R^{d1} represents an alkyl group or an aryl group.

R^{a1} and R^{a2} may bond to each other to form a ring. More specifically, R^{a1} and R^{a2} may bond to each other via a single bond or a linking group to form a ring. Examples of the linking group include —O—, —S—, an alkylene group, a silylene group, an alkenylene group, a cycloalkylene group, a cycloalkenylene group, an arylene group, a divalent heterocyclic group, and an imino group.

In a case where R^{a1} and R^{a2} bond to each other to form a ring, as a result, the carbon atoms contained in the structure including R^{a1} and R^{a2} is 4 or more.

Examples of the ring formed by R^{a1} and R^{a2} boding to each other include an aromatic ring (an aromatic hydrocarbon ring or an aromatic heterocyclic ring) and a non-aromatic ring.

Examples of the aromatic ring include a benzene ring and a fluorene ring.

In a case where R^{a1} and R^{a2} bond to each other to form a ring, the specific compound is preferably a compound represented by Formula (1-3).

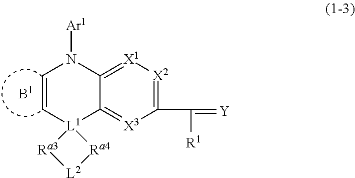

In Formula (1-3), the definitions of Ar¹, X¹ to X³, R¹, L¹, B¹, and Y are as described above.

R^{a3} and R^{a4} each independently represent a divalent substituent having 2 or more carbon atoms.

The carbon atoms contained in the divalent substituent having 2 or more carbon atoms is preferably 3 or more from the viewpoint of obtaining a superior effect of the present invention. That is, the divalent substituent having 2 or more carbon atoms is preferably a divalent substituent having 3 or more carbon atoms. The upper limit of the carbon atoms is not particularly limited, but is 10 or less, for example.

Examples of the divalent substituent having 2 or more carbon atoms include a divalent aliphatic hydrocarbon group having 2 or more carbon atoms, which may contain a hetero atom, and a divalent aromatic group having 2 or more carbon atoms.

Examples of the aliphatic hydrocarbon group include an alkylene group, an alkenylene group, and an alkynylene group. These groups may contain a hetero atom such as an oxygen atom, a nitrogen atom, and a sulfur atom.

Examples of the aromatic groups include an arylene group and a heteroarylene group.

Among these, from the viewpoint of obtaining a superior effect of the present invention, an arylene group having 3 or more carbon atoms (for example, a phenylene group) or a heteroarylene group having 3 or more carbon atoms is preferable.

L² represents a single bond or a linking group (a divalent linking group). Examples of the linking group include —O—, —S—, an alkylene group, a silylene group, an alkenylene group, a cycloalkylene group, a cycloalkenylene group, an arylene group, a divalent heterocyclic group, or an imino group.

The specific compound has none of a carboxy group, a salt of a carboxy group, a phosphoric acid group, a salt of a phosphoric acid group, a sulfonic acid group, or a salt of a sulfonic acid group from the viewpoint of avoiding deterioration in the vapor deposition suitability.

More specifically, the substituent in the specific compound has none of a carboxy group, a salt of a carboxy group, a phosphoric acid group, a salt of a phosphoric acid group, a sulfonic acid group, or a salt of a sulfonic acid group.

From the viewpoint of obtaining a superior effect of the present invention, the specific compound is preferably the compound represented by Formula (2), more preferably the compound represented by Formula (3), and still more preferably the compound represented by Formula (4).

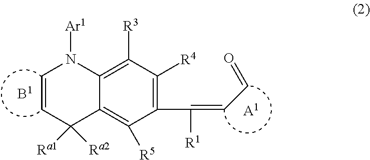

In Formula (2), the definitions of Ar¹, R¹, R^{a1}, R^{a2}, B¹, and A¹ are as described above.

R³ to R⁵ each independently represent a hydrogen atom or a substituent. The definition of the substituent is synonymous with the substituent W described below.

The compound represented by Formula (2) has none of a carboxy group, a salt of a carboxy group, a phosphoric acid group, a salt of a phosphoric acid group, a sulfonic acid group, or a salt of a sulfonic acid group.

In a case where R^{a1} and R^{a2} form a ring, the compound represented by Formula (2) is preferably a compound represented by Formula (2-1).

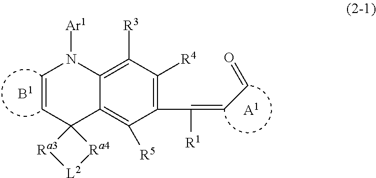

In Formula (2-1), the definitions of Ar¹, R¹, B¹, R³ to R⁵, and A¹ are as described above.

In Formula (2-1), the definitions of R^{a3}, R^{a4}, and L² are the same as the definition of each group in Formula (1-3).

The compound represented by Formula (2-1) has none of a carboxy group, a salt of a carboxy group, a phosphoric acid group, a salt of a phosphoric acid group, a sulfonic acid group, or a salt of a sulfonic acid group.

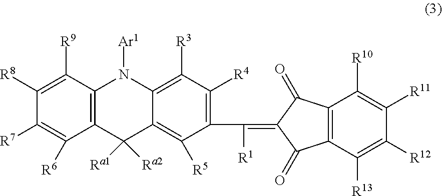

In Formula (3), the definitions of Ar¹, R¹, R³ to R⁵, R^{a1}, and R^{a2} are as described above.

R⁶ to R¹³ each independently represent a hydrogen atom or a substituent. The definition of the substituent is synonymous with the substituent W described below.

R¹⁰ and R¹¹, R¹¹ and R¹², and R¹² and R¹³ may respectively independently bond to each other to form a ring. Examples of the type of formed ring include an aromatic ring (an aromatic hydrocarbon ring or an aromatic heterocyclic ring) and a non-aromatic ring. Examples of the aromatic ring include a benzene ring and a fluorene ring.

The compound represented by Formula (3) has none of a carboxy group, a salt of a carboxy group, a phosphoric acid group, a salt of a phosphoric acid group, a sulfonic acid group, or a salt of a sulfonic acid group.

In a case where R^{a1} and R^{a2} form a ring, the compound represented by Formula (3) is preferably a compound represented by Formula (3-1).

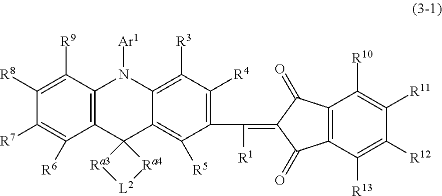

In Formula (3-1), the definitions of Ar¹, R¹, and R³ to R¹³ and are as described above.

In Formula (3-1), the definitions of R^{a3}, R^{a4}, and L² are the same as the definition of each group in Formula (1-3).

The compound represented by Formula (3-1) has none of a carboxy group, a salt of a carboxy group, a phosphoric acid group, a salt of a phosphoric acid group, a sulfonic acid group, or a salt of a sulfonic acid group.

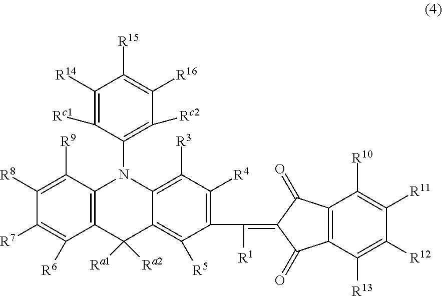

In Formula (4), the definitions of R, R³ to R¹³, R^{a1}, and R^{a2} are as described above.

R¹⁴ to R¹⁶ each independently represent a hydrogen atom or a substituent. The definition of the substituent is synonymous with the substituent W described below.

R^{c1} and R^{c2} each independently represent a hydrogen atom or a substituent, and at least one of R^{c1} or R^{c2} represents a substituent. The definition of the substituent is synonymous with the substituent W described below. Examples of the substituent include an alkyl group, an aryl group, and a heteroaryl group.

The compound represented by Formula (4) has none of a carboxy group, a salt of a carboxy group, a phosphoric acid group, a salt of a phosphoric acid group, a sulfonic acid group, or a salt of a sulfonic acid group.

In a case where R^{a1} and R^{a2} form a ring, the compound represented by Formula (4) is preferably a compound represented by Formula (4-1).

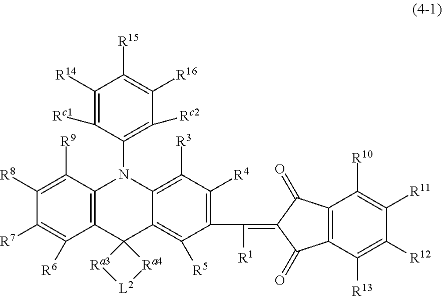

In Formula (4-1), the definitions of R^{c1}, R^{c2}, R¹, and R³ to R¹⁶ and are as described above.

In Formula (4-1), the definitions of R^{a3}, R^{a4}, and L² are the same as the definition of each group in Formula (1-3).

The compound represented by Formula (4-1) has none of a carboxy group, a salt of a carboxy group, a phosphoric acid group, a salt of a phosphoric acid group, a sulfonic acid group, or a salt of a sulfonic acid group.

(Substituent W)

The substituent W in the present specification will be described below.

Examples of the substituent W include a halogen atom (such as a fluorine atom, a chlorine atom, a bromine atom, and an iodine atom), an alkyl group (including a cycloalkyl group, a bicycloalkyl group, and a tricycloalkyl group), an alkenyl group (including a cycloalkenyl group and a bicycloalkenyl group), an alkynyl group, an aryl group, a heterocyclic group (including a heteroaryl group), a cyano group, a hydroxy group, a nitro group, an alkoxy group, an aryloxy group, a silyloxy group, a heterocyclic oxy group, an acyloxy group, a carbamoyloxy group, an alkoxycarbonyloxy group, an aryloxycarbonyloxy group, an amino group (including an anilino group), an ammonium group, an acylamino group, an aminocarbonylamino group, an alkoxycarbonylamino group, an aryloxycarbonylamino group, a sulfamoylamino group, an alkyl- or arylsulfonylamino group, a mercapto group, an alkylthio group, an arylthio group, a heterocyclic thio group, a sulfamoyl group, an alkyl- or arylsulfinyl group, an alkyl- or arylsulfonyl group, an acyl group, an aryloxycarbonyl group, an alkoxycarbonyl group, a carbamoyl group, an aryl- or heterocyclic azo group, an imide group, a phosphino group, a phosphinyl group, a phosphinyloxy group, a phosphinylamino group, a phosphono group, a silyl group, a hydrazino group, a ureido group, and a boronic acid group (—B(OH)₂).

Also, the substituent W may be further substituted with the substituent W. For example, an alkyl group may be substituted with a halogen atom.

The specific compounds are exemplified below, but the specific compounds according to the embodiment of the present invention are not limited thereto.

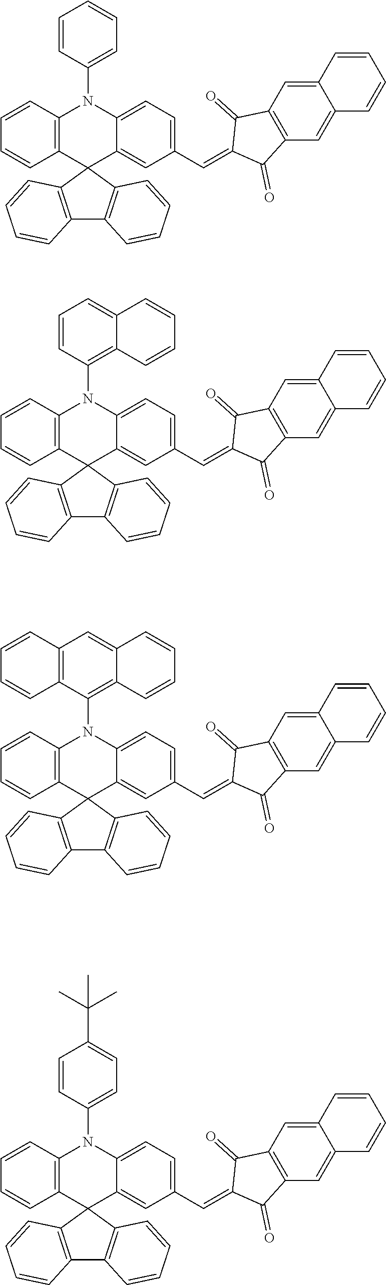 [SMILES: O=C1C(=Cc2ccc3c(c2)C2(c4ccccc4-c4ccccc42)c2ccccc2N3c2ccccc2)C(=O)c2cc3ccccc3cc21 ; O=C1C(=Cc2ccc3c(c2)C2(c4ccccc4-c4ccccc42)c2ccccc2N3c2cccc3ccccc23)C(=O)c2cc3ccccc3cc21 ; O=C1C(=Cc2ccc3c(c2)C2(c4ccccc4-c4ccccc42)c2ccccc2N3c2c3ccccc3cc3ccccc23)C(=O)c2cc3ccccc3cc21 ; CC(C)(C)c1ccc(N2c3ccccc3C3(c4ccccc4-c4ccccc43)c3cc(C=C4C(=O)c5cc6ccccc6cc5C4=O)ccc32)cc1]

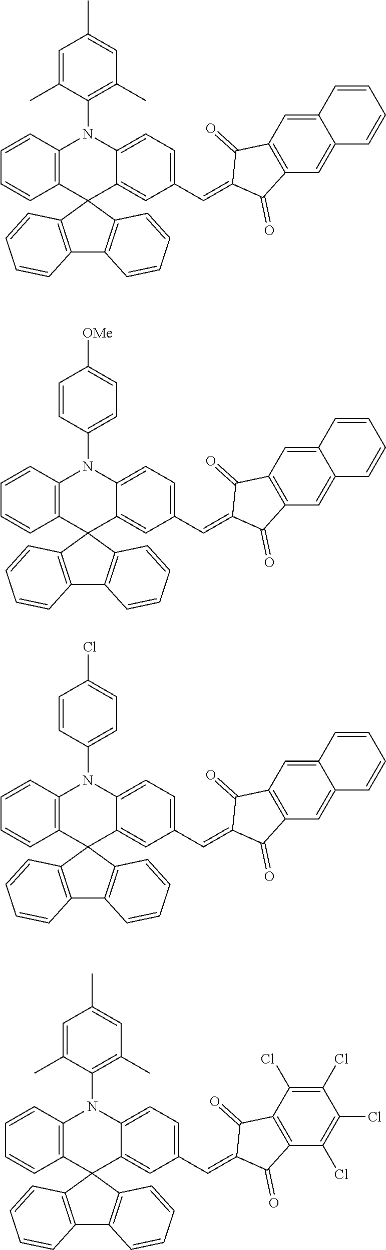 [SMILES: Cc1cc(C)c(N2c3ccccc3C3(c4ccccc4-c4ccccc43)c3cc(C=C4C(=O)c5cc6ccccc6cc5C4=O)ccc32)c(C)c1 ; COc1ccc(N2c3ccccc3C3(c4ccccc4-c4ccccc43)c3cc(C=C4C(=O)c5cc6ccccc6cc5C4=O)ccc32)cc1 ; O=C1C(=Cc2ccc3c(c2)C2(c4ccccc4-c4ccccc42)c2ccccc2N3c2ccc(Cl)cc2)C(=O)c2cc3ccccc3cc21 ; Cc1cc(C)c(N2c3ccccc3C3(c4ccccc4-c4ccccc43)c3cc(C=C4C(=O)c5c(Cl)c(Cl)c(Cl)c(Cl)c5C4=O)ccc32)c(C)c1]

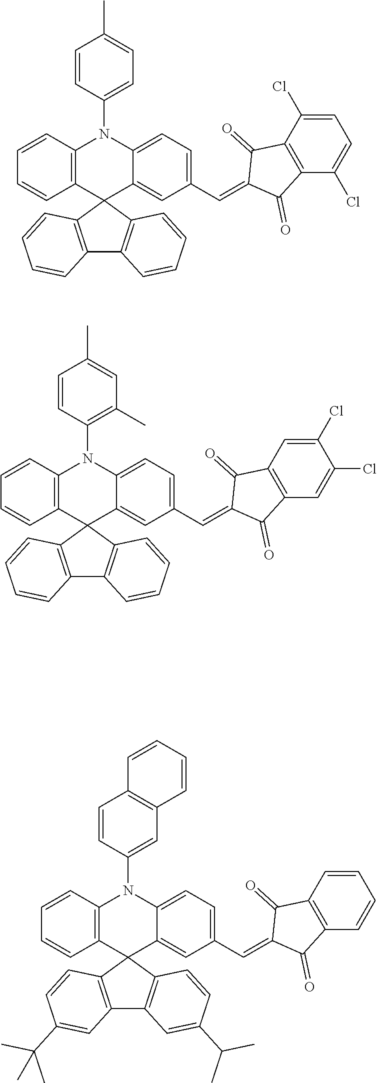 [SMILES: Cc1ccc(N2c3ccccc3C3(c4ccccc4-c4ccccc43)c3cc(C=C4C(=O)c5c(Cl)ccc(Cl)c5C4=O)ccc32)cc1 ; Cc1ccc(N2c3ccccc3C3(c4ccccc4-c4ccccc43)c3cc(C=C4C(=O)c5cc(Cl)c(Cl)cc5C4=O)ccc32)c(C)c1 ; CC(C)c1ccc2c(c1)-c1cc(C(C)(C)C)ccc1C21c2ccccc2N(c2ccc3ccccc3c2)c2ccc(C=C3C(=O)c4ccccc4C3=O)cc21]

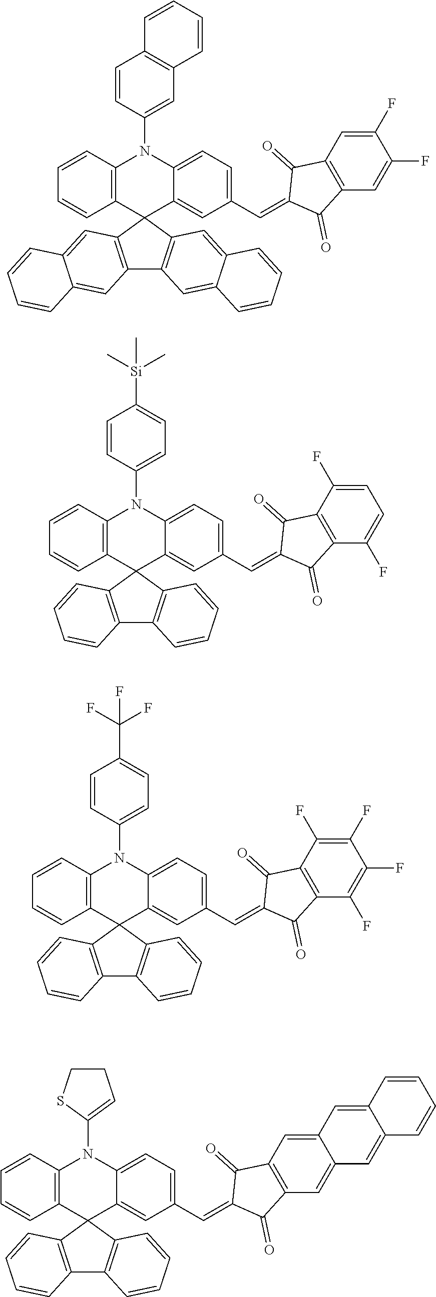 [SMILES: O=C1C(=Cc2ccc3c(c2)C2(c4cc5ccccc5cc4-c4cc5ccccc5cc42)c2ccccc2N3c2ccc3ccccc3c2)C(=O)c2cc(F)c(F)cc21 ; C[Si](C)(C)c1ccc(N2c3ccccc3C3(c4ccccc4-c4ccccc43)c3cc(C=C4C(=O)c5c(F)ccc(F)c5C4=O)ccc32)cc1 ; O=C1C(=Cc2ccc3c(c2)C2(c4ccccc4-c4ccccc42)c2ccccc2N3c2ccc(C(F)(F)F)cc2)C(=O)c2c(F)c(F)c(F)c(F)c21 ; O=C1C(=Cc2ccc3c(c2)C2(c4ccccc4-c4ccccc42)c2ccccc2N3C2=CCCS2)C(=O)c2cc3cc4ccccc4cc3cc21]

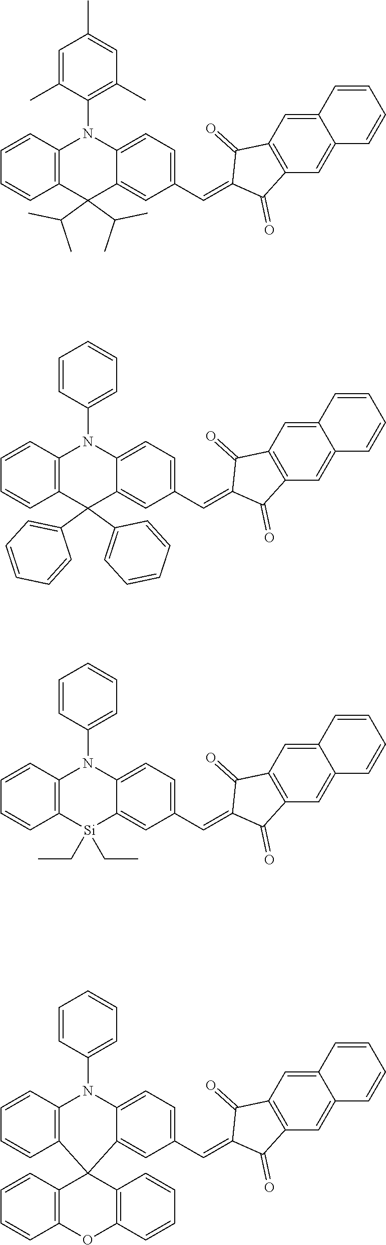 [SMILES: Cc1cc(C)c(N2c3ccccc3C(C(C)C)(C(C)C)c3cc(C=C4C(=O)c5cc6ccccc6cc5C4=O)ccc32)c(C)c1 ; O=C1C(=Cc2ccc3c(c2)C(c2ccccc2)(c2ccccc2)c2ccccc2N3c2ccccc2)C(=O)c2cc3ccccc3cc21 ; CC[Si]1(CC)c2ccccc2N(c2ccccc2)c2ccc(C=C3C(=O)c4cc5ccccc5cc4C3=O)cc21 ; O=C1C(=Cc2ccc3c(c2)C2(c4ccccc4Oc4ccccc42)c2ccccc2N3c2ccccc2)C(=O)c2cc3ccccc3cc21]

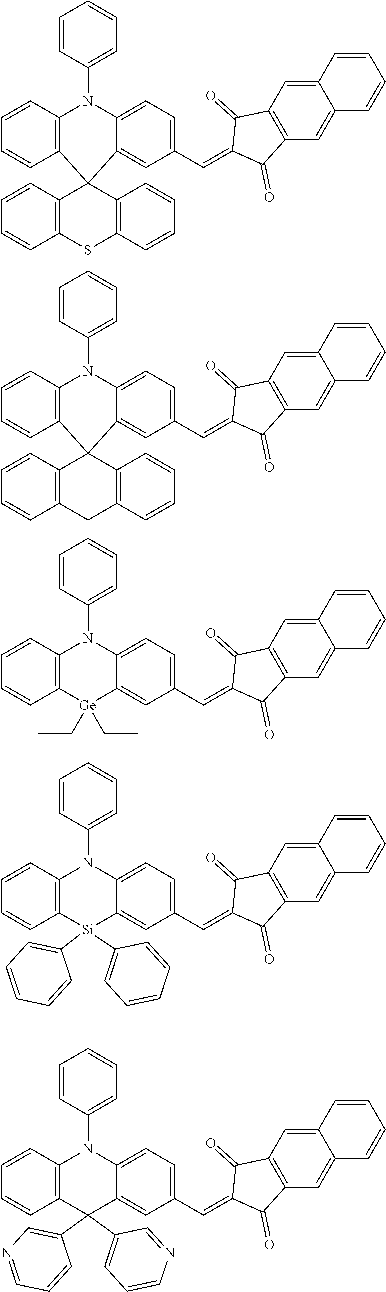

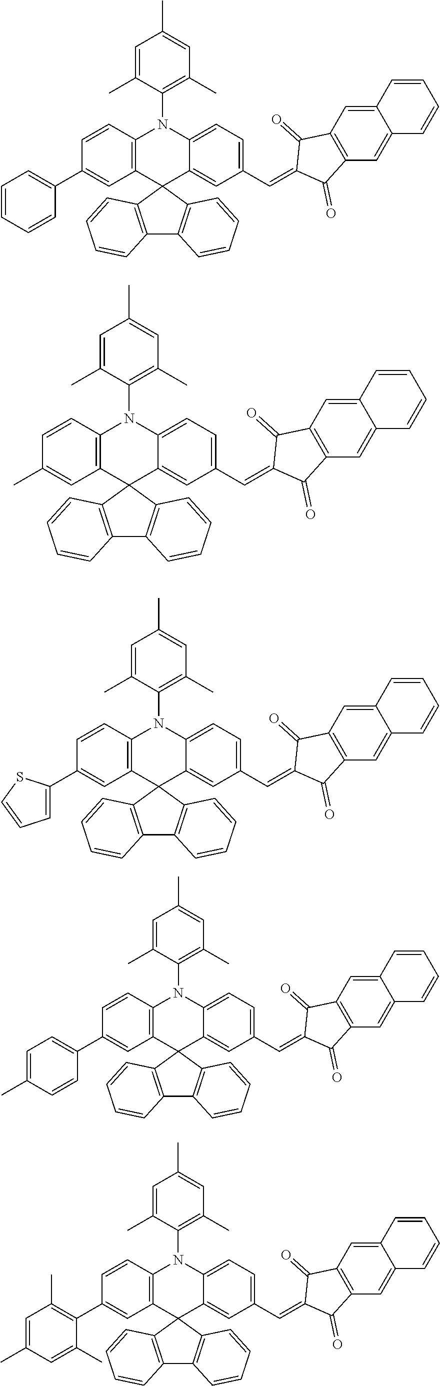

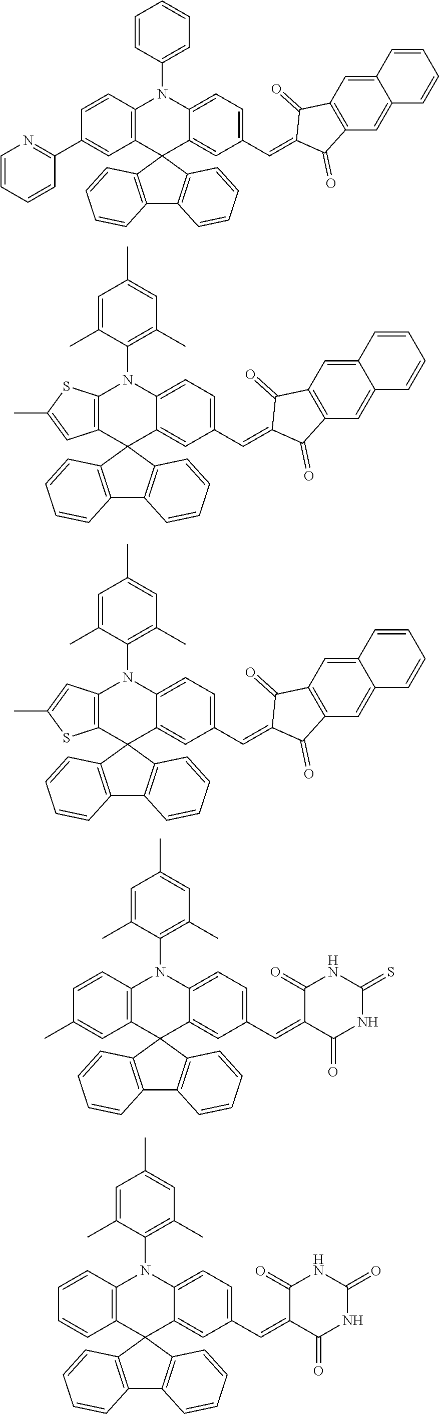

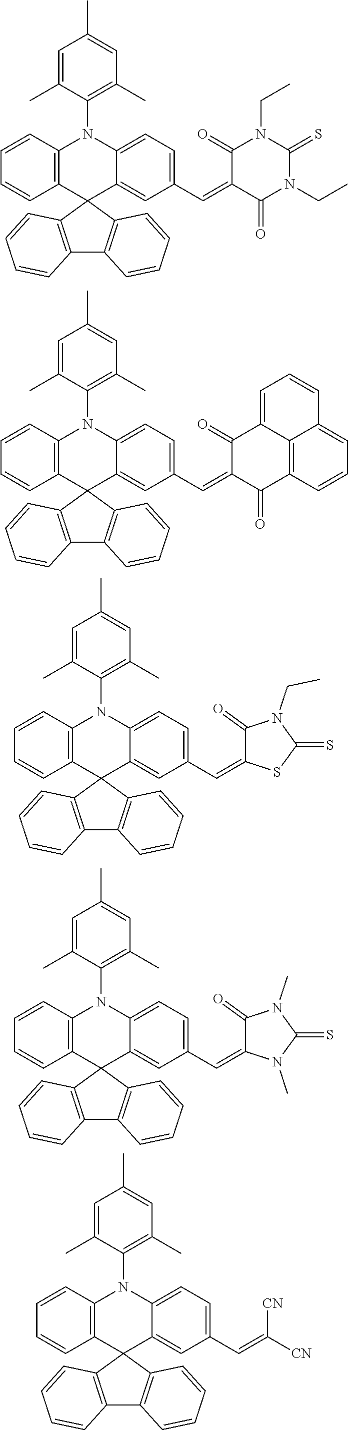

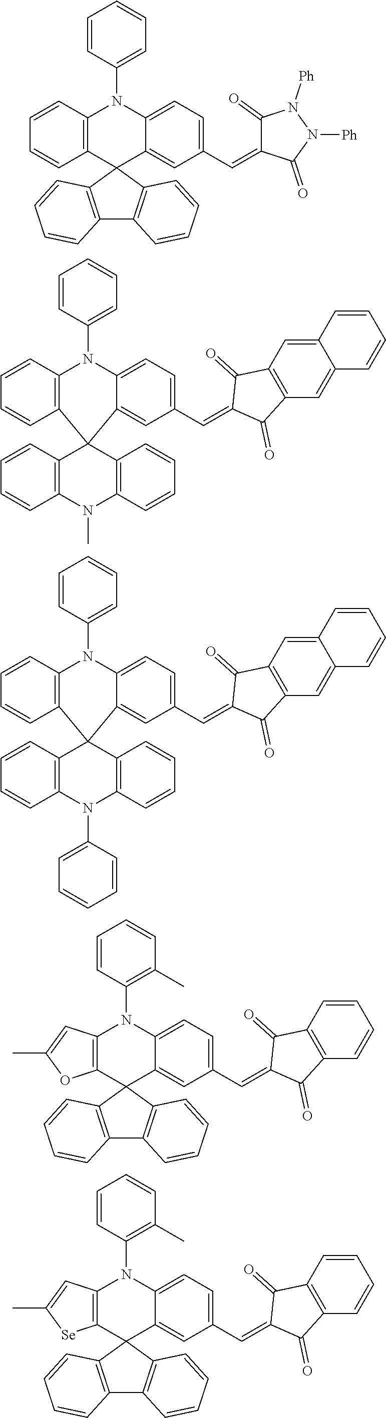

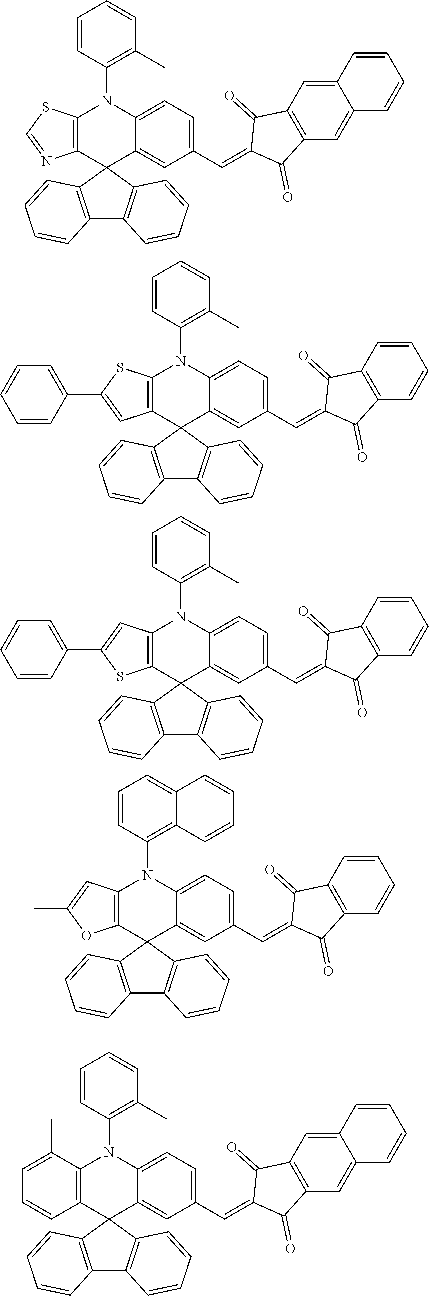

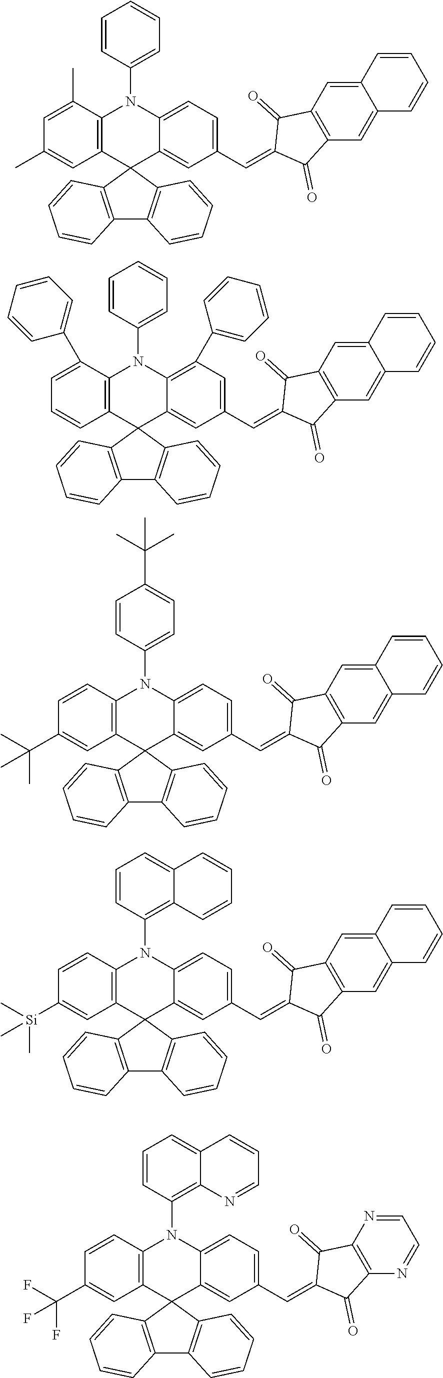

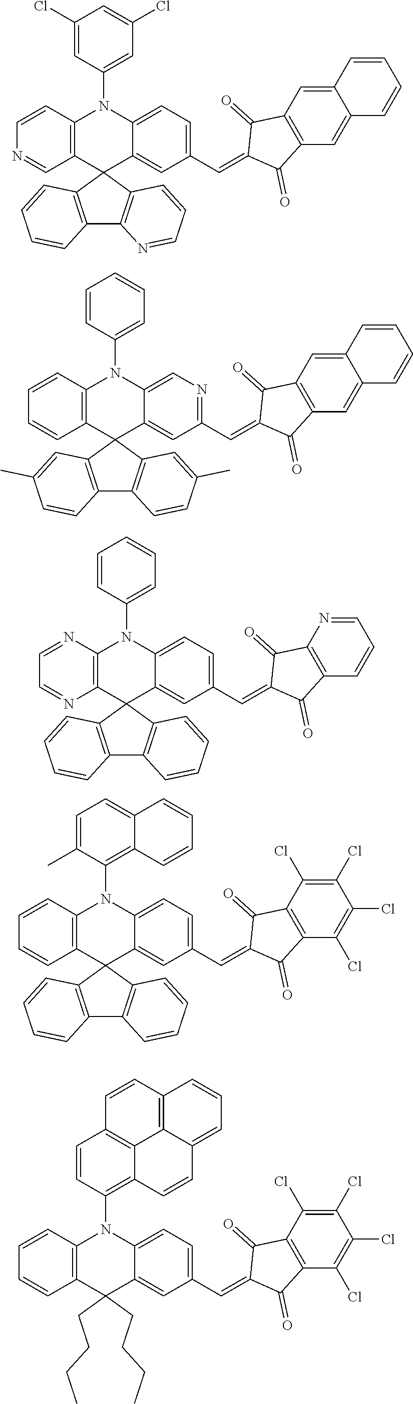

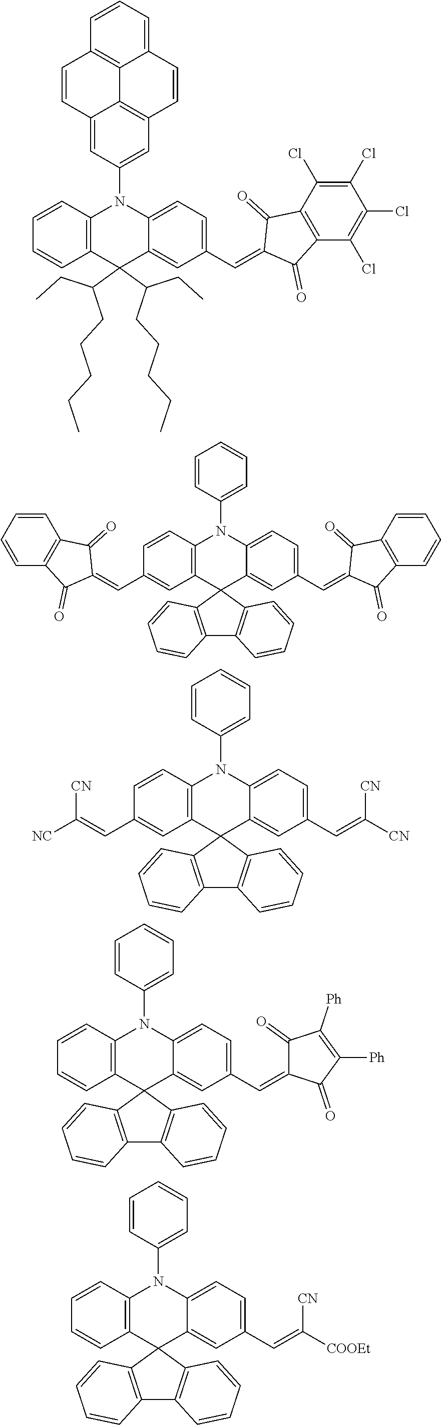

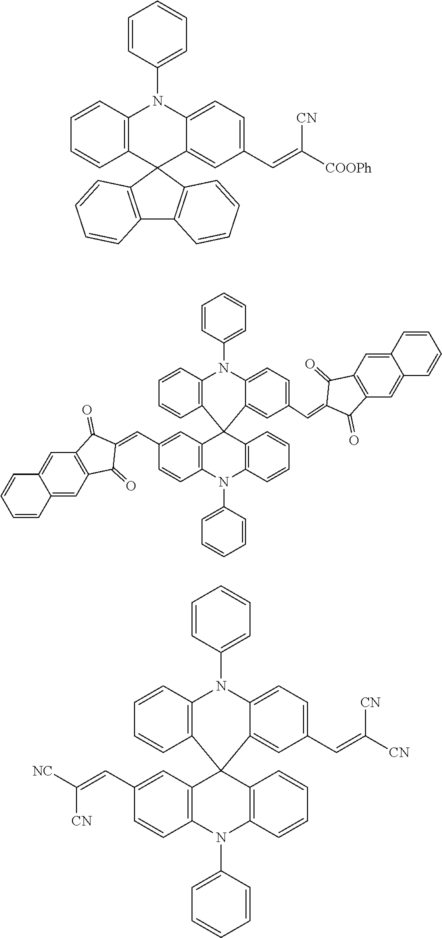

The specific compound is particularly useful as a material of the photoelectric conversion film used for the imaging element, the optical sensor, or a photoelectric cell. In addition, the specific compound usually functions as the p-type organic semiconductor in the photoelectric conversion film in many cases. The specific compound can also be used as a coloring material, a liquid crystal material, an organic semiconductor material, a charge transport material, a pharmaceutical material, and a fluorescent diagnostic material.

The specific compound is preferably a compound in which an ionization potential in a single film is −5.0 to −6.0 eV from the viewpoints of stability in a case of using the compound as the p-type organic semiconductor and matching of energy levels between the compound and the n-type organic semiconductor.

The maximum absorption wavelength of the specific compound is not particularly limited, but is preferably in the range of 510 to 570 nm, and more preferably in the range of 520 to 560 nm, from the point that the photoelectric conversion film in the photoelectric conversion element according to the embodiment of the present invention is suitably used as an organic photoelectric conversion film that receives (absorbs) green light and performs photoelectrically conversion.

The absorption half-width of the specific compound is not particularly limited, but is preferably 95 nm or less, more preferably 90 nm or less, and still more preferably 85 nm or less, from the point that the photoelectric conversion film in the photoelectric conversion element according to the embodiment of the present invention is suitably used as an organic photoelectric conversion film that receives (absorbs) green light and performs photoelectrically conversion. The lower limit is not particularly limited, but is often 60 nm or more.

The maximum absorption wavelength and the absorption half-width are values measured in a state of a film of a specific compound (for example, a vapor deposited film of the specific compound).

The maximum absorption wavelength of the photoelectric conversion film is not particularly limited, but is preferably in the range of 510 to 570 nm, and more preferably in the range of 520 to 560 nm, from the point that the photoelectric conversion film in the photoelectric conversion element according to the embodiment of the present invention is suitably used as an organic photoelectric conversion film that receives (absorbs) green light and performs photoelectrically conversion.

<n-Type Organic Semiconductor>

It is preferable that the photoelectric conversion film contains the n-type organic semiconductor as a component other than the specific compound.

The n-type organic semiconductor is an acceptor-property organic semiconductor material (a compound), and refers to an organic compound having a property of easily accepting an electron. More specifically, the n-type organic semiconductor refers to an organic compound having a large electron affinity of two organic compounds used in contact with each other.

Examples of the n-type organic semiconductor include a condensed aromatic carbocyclic compound (for example, fullerene, a naphthalene derivative, an anthracene derivative, a phenanthrene derivative, a tetracene derivative, a pyrene derivative, a perylene derivative, and a fluoranthene derivative); a 5 to 7 membered heterocyclic compound having at least one of a nitrogen atom, an oxygen atom, or a sulfur atom (for example, pyridine, pyrazine, pyrimidine, pyridazine, triazine, quinoline, quinoxaline, quinazoline, phthalazine, cinnoline, isoquinoline, pteridine, acridine, phenazine, phenanthroline, tetrazole, pyrazole, imidazole, and thiazole); a polyarylene compound; a fluorene compound; a cyclopentadiene compound; a silyl compound; and a metal complex having a nitrogen-containing heterocyclic compound as the ligands.

An organic dye may be used as the n-type organic semiconductor. Examples of the organic dye include a cyanine dye, a styryl dye, a hemicyanine dye, a merocyanine dye (including zeromethine merocyanine (simple merocyanine)), a rhodacyanine dye, an allopolar dye, an oxonol dye, a hemioxonol dye, a squarylium dye, a croconium dye, an azamethine dye, a coumarin dye, an arylidene dye, an anthraquinone dye, a triphenylmethane dye, an azo dye, an azomethine dye, a metallocene dye, a fluorenone dye, a flugide dye, a perylene dye, a phenazine dye, a phenothiazine dye, a quinone dye, a diphenylmethane dye, a polyene dye, an acridine dye, an acridinone dye, a diphenylamine dye, a quinophthalone dye, a phenoxazine dye, a phthaloperylene dye, a dioxane dye, a porphyrin dye, a chlorophyll dye, a phthalocyanine dye, a subphthalocyanine dye, and a metal complex dye.

The molecular weight of the n-type organic semiconductor is preferably 200 to 1200, and more preferably 200 to 900.

From the point that the photoelectric conversion film in the photoelectric conversion element according to the embodiment of the present invention is suitably used as an organic photoelectric conversion film that receives (absorbs) green light and performs photoelectrically conversion, it is preferable that the n-type organic semiconductor is colorless or has a maximum absorption wavelength and/or an absorption waveform close to that of the specific compound, and as the specific value, the maximum absorption wavelength of the n-type organic semiconductor is preferably 400 nm or less or in the range of 500 to 600 nm.

It is preferable that the photoelectric conversion film has a bulk hetero structure formed in a state in which the specific compound and the n-type organic semiconductor are mixed. The bulk hetero structure refers to a layer in which the specific compound and the n-type organic semiconductor are mixed and dispersed in the photoelectric conversion film. The photoelectric conversion film having the bulk hetero structure can be formed by either a wet method or a dry method. The bulk hetero structure is described in detail in, for example, paragraphs [0013] to [0014] of JP2005-303266A.

From the viewpoint of responsiveness of the photoelectric conversion element, the content of the specific compound to the total content of the specific compound and the n-type organic semiconductor (=film thickness in terms of single layer of specific compound/(film thickness in terms of single layer of specific compound+film thickness in terms of single layer of n-type organic semiconductor)×100) is preferably 20 to 80 volume %, and more preferably 40 to 80 volume %.

It is preferable that the photoelectric conversion film is substantially formed of the specific compound and the n-type organic semiconductor. The term “substantially” means that the total content of the specific compound and the n-type organic semiconductor to the total mass of the photoelectric conversion film is 95 mass % or more.

The photoelectric conversion film containing the specific compound is a non-luminescent film, and has a feature different from an organic light emitting diode (OLED). The non-luminescent film means a film having a light emission quantum efficiency of 1% or less, and the luminescence quantum efficiency is preferably 0.5% or less, and more preferably 0.1% or less.

<Film Formation Method>

The photoelectric conversion film can be formed mostly by a dry film formation method. Examples of the dry film formation method include a physical vapor deposition method such as a vapor deposition method (in particular, a vacuum evaporation method), a sputtering method, an ion plating method, and molecular beam epitaxy (MBE), and chemical vapor deposition (CVD) such as plasma polymerization. Among these, the vacuum evaporation method is preferable. In a case where the photoelectric conversion film is formed by the vacuum evaporation method, a producing condition such as a degree of vacuum and a vapor deposition temperature can be set according to the normal method.

The thickness of the photoelectric conversion film is preferably 10 to 1000 nm, more preferably 50 to 800 nm, still more preferably 50 to 500 nm, and particularly preferably 50 to 300 nm.

<Electrode>

The electrode (the upper electrode (the transparent conductive film) 15 and the lower electrode (the conductive film) 11) is formed of a conductive material. Examples of the conductive material include metals, alloys, metal oxides, electrically conductive compounds, and mixtures thereof.

Since light is incident through the upper electrode 15, the upper electrode 15 is preferably transparent to light to be detected. Examples of the material forming the upper electrode 15 include conductive metal oxides such as tin oxide (ATO, FTO) doped with antimony, fluorine, or the like, tin oxide, zinc oxide, indium oxide, indium tin oxide (ITO), and indium zinc oxide (IZO); metal thin films such as gold, silver, chromium, and nickel; mixtures or laminates of these metals and the conductive metal oxides; and organic conductive materials such as polyaniline, polythiophene, and polypyrrole. Among these, conductive metal oxides are preferable from the viewpoints of high conductivity, transparency, and the like.

In general, in a case where the conductive film is made to be thinner than a certain range, a resistance value is rapidly increased. However, in the solid-state imaging element into which the photoelectric conversion element according to the present embodiment is incorporated, the sheet resistance is preferably 100 to 10000Ω/□, and the degree of freedom of the range of the film thickness that can be thinned is large. In addition, as the thickness of the upper electrode (the transparent conductive film) 15 is thinner, the amount of light that the upper electrode absorbs becomes smaller, and the light transmittance usually increases. The increase in the light transmittance causes an increase in light absorbance in the photoelectric conversion film and an increase in the photoelectric conversion ability, which is preferable. Considering the suppression of leakage current, an increase in the resistance value of the thin film, and an increase in transmittance accompanied by the thinning, the film thickness of the upper electrode 15 is preferably 5 to 100 nm, and more preferably 5 to 20 nm.

There is a case where the lower electrode 11 has transparency or an opposite case where the lower electrode does not have transparency and reflects light, depending on the application. Examples of a material constituting the lower electrode 11 include conductive metal oxides such as tin oxide (ATO, FTO) doped with antimony, fluorine, or the like, tin oxide, zinc oxide, indium oxide, indium tin oxide (ITO), and indium zinc oxide (IZO); metals such as gold, silver, chromium, nickel, titanium, tungsten, and aluminum, conductive compounds (for example, titanium nitride (TiN)) such as oxides or nitrides of these metals; mixtures or laminates of these metals and conductive metal oxides; and organic conductive materials such as polyaniline, polythiophene, and polypyrrole.

The method of forming electrodes is not particularly limited, and can be appropriately selected in accordance with the electrode material. Specific examples thereof include a wet method such as a printing method and a coating method; a physical method such as a vacuum evaporation method, a sputtering method, and an ion plating method; and a chemical method such as a CVD method and a plasma CVD method.

In a case where the material of the electrode is ITO, examples thereof include an electron beam method, a sputtering method, a resistance thermal vapor deposition method, a chemical reaction method (such as a sol-gel method), and a coating method with a dispersion of indium tin oxide.

<Charge Blocking Film: Electron Blocking Film and Positive Hole Blocking Film>

It is also preferable that the photoelectric conversion element according to the embodiment of the present invention has one or more interlayers between the conductive film and the transparent conductive film, in addition to the photoelectric conversion film. An example of the interlayer includes the charge blocking film. In the case where the photoelectric conversion element has this film, the characteristics (such as photoelectric conversion efficiency and responsiveness) of the photoelectric conversion element to be obtained become superior. Examples of the charge blocking film include the electron blocking film and the positive hole blocking film. Hereinafter, the films will be described in detail.

(Electron Blocking Film)

The electron blocking film includes an electron donating compound.

Specific examples of a low molecular material include aromatic diamine compounds such as N,N′-bis(3-methylphenyl)-(1,1′-biphenyl)-4,4′-diamine (TPD) and 4,4′-bis[N-(naphthyl)-N-phenyl-amino] biphenyl (α-NPD); porphyrin compounds such as porphyrin, copper tetraphenylporphyrin, phthalocyanine, copper phthalocyanine, and titanium phthalocyanine oxide; and oxazole, oxadiazole, triazole, imidazole, imidazolone, a stilbene derivative, a pyrazoline derivative, tetrahydroimidazole, polyarylalkane, butadiene, 4,4′,4″-tris(N-(3-methylphenyl)-N-phenylamino) triphenylamine (m-MTDATA), a triazole derivative, an oxadiazole derivative, an imidazole derivative, a polyarylalkane derivative, a pyrazoline derivative, a pyrazolone derivative, a phenylenediamine derivative, an arylamine derivative, an amino-substituted chalcone derivative, an oxazole derivative, a styrylanthracene derivative, a fluorenone derivative, a hydrazone derivative, and a silazane derivative.

Specific examples of a polymer material include a polymer such as phenylenevinylene, fluorene, carbazole, indole, pyrene, pyrrole, picoline, thiophene, acetylene, and diacetylene, and a derivative thereof. In addition, compounds described in paragraphs [0049] to [0063] of JP5597450B, compounds described in paragraphs [0119] to [0158] of JP2011-225544A, and compounds described in paragraphs [0086] to [0090] of JP2012-094660A are exemplified.

The electron blocking film may be configured by a plurality of films.

The electron blocking film may be formed of an inorganic material. In general, an inorganic material has a dielectric constant larger than that of an organic material. Therefore, in a case where the inorganic material is used in the electron blocking film, a large voltage is applied to the photoelectric conversion film. Therefore, the photoelectric conversion efficiency increases. Examples of the inorganic material that can be used in the electron blocking film include calcium oxide, chromium oxide, copper chromium oxide, manganese oxide, cobalt oxide, nickel oxide, copper oxide, copper gallium oxide, copper strontium oxide, niobium oxide, molybdenum oxide, copper indium oxide, silver indium oxide, and iridium oxide.

(Positive Hole Blocking Film)

The positive hole blocking film includes an electron accepting compound.

Examples of the electron accepting compound include an oxadiazole derivative such as 1,3-bis(4-tert-butylphenyl-1,3,4-oxadiazolyl)phenylene (OXD-7); an anthraquinodimethane derivative; a diphenylquinone derivative; bathocuproine, bathophenanthroline, and derivatives thereof; a triazole compound; a tris(8-hydroxyquinolinato)aluminum complex; a bis(4-methyl-8-quinolinato)aluminum complex; a distyrylarylene derivative; and a silole compound. In addition, compounds described in paragraphs [0056] to [0057] of JP2006-100767A are exemplified.

The method of producing the charge blocking film is not particularly limited, but a dry film formation method and a wet film formation method are exemplified. Examples of the dry film formation method include a vapor deposition method and a sputtering method. The vapor deposition method may be any of physical vapor deposition (PVD) method and chemical vapor deposition (CVD) method, and physical vapor deposition method such as vacuum evaporation method is preferable. Examples of the wet film formation method include an inkjet method, a spray method, a nozzle printing method, a spin coating method, a dip coating method, a casting method, a die coating method, a roll coating method, a bar coating method, and a gravure coating method, and an inkjet method is preferable from the viewpoint of high precision patterning.

Each thickness of the charge blocking films (the electron blocking film and the positive hole blocking film) is preferably 3 to 200 nm, more preferably 5 to 100 nm, and still more preferably 5 to 30 nm.

<Substrate>

The photoelectric conversion element may further include a substrate. The type of substrate to be used is not particularly limited, but a semiconductor substrate, a glass substrate, and a plastic substrate are exemplified.

The position of the substrate is not particularly limited, but in general, the conductive film, the photoelectric conversion film, and the transparent conductive film are laminated on the substrate in this order.

<Sealing Layer>

The photoelectric conversion element may further include a sealing layer. The performance of the photoelectric conversion material may deteriorate noticeably due to the presence of deterioration factors such as water molecules. The deterioration can be prevented by sealing and coating the entirety of the photoelectric conversion film with the sealing layer such as diamond-like carbon (DLC) or ceramics such as metal oxide, or metal nitride, and metal nitride oxide which are dense and into which water molecules do not permeate.

The material of the sealing layer may be selected and the sealing layer may be produced according to the description in paragraphs [0210] to [0215] of JP2011-082508A.

<Imaging Element>

An example of the application of the photoelectric conversion element includes an imaging element. The imaging element is an element that converts optical information of an image into an electric signal, and usually includes a plurality of photoelectric conversion elements.

FIG. 3 is a schematic cross-sectional view showing a schematic configuration of an imaging element for describing an embodiment of the present invention. This imaging element is mounted on an imaging element such as a digital camera and a digital video camera, an electronic endoscope, and imaging modules such as a cellular phone.

An imaging element 20a shown in FIG. 3 includes a photoelectric conversion element 10a according to the embodiment of the present invention, a blue photoelectric conversion element 22, and a red photoelectric conversion element 24, which are laminated along the light incident direction. As described above, the photoelectric conversion element 10a can mainly function as a green photoelectric conversion element capable of receiving green light.

The imaging element 20a is a so-called laminated type color separation imaging element. The photoelectric conversion element 10a, the blue photoelectric conversion element 22, and the red photoelectric conversion element 24 have different wavelength spectra to be detected. That is, the blue photoelectric conversion element 22 and the red photoelectric conversion element 24 correspond to photoelectric conversion elements that receive (absorb) light having a wavelength different from the light received by the photoelectric conversion element 10a. The photoelectric conversion element 10a can receive green light, the blue photoelectric conversion element 22 can receive blue light, and the red photoelectric conversion element can receive red light.

Green light means light in the wavelength range of 500 to 600 nm, blue light means light in the wavelength range of 400 to 500 nm, and red light means light in the wavelength range of 600 to 700 nm.

In a case where light is incident on the imaging element 20a in the direction of the arrow, first, green light is absorbed by the photoelectric conversion element 10a, but blue light and red light are transmitted through the photoelectric conversion element 10a. In a case where the light transmitted through the photoelectric conversion element 10a travels to the blue photoelectric conversion element 22, the blue light is absorbed, but the red light is transmitted through the blue photoelectric conversion element 22. Then, light transmitted through the blue photoelectric conversion element 22 is absorbed by the red photoelectric conversion element 24. As described above, in the imaging element 20a, which is a laminated type color separation imaging element, one pixel can be configured with three light receiving sections of green, blue, and red, and a large area of the light receiving section can be taken.

In particular, as described above, the photoelectric conversion element 10a according to the embodiment of the present invention has a narrow absorption peak half-width, and thus absorptions of blue light and red light do not occur, and it is difficult to affect the detectability of the blue photoelectric conversion element 22 and the red photoelectric conversion element 24.

The configuration of the blue photoelectric conversion element 22 is not particularly limited, but examples thereof include a photoelectric conversion element having a conductive film, a blue photoelectric conversion film, and a transparent conductive film in this order.

The type of the blue photoelectric conversion film is not particularly limited as long as it is a photoelectric conversion film capable of receiving blue light, and includes an organic blue photoelectric conversion film or an inorganic blue photoelectric conversion film, and an organic blue photoelectric conversion film (a blue photoelectric conversion film composed of an organic compound) is preferable.

The maximum absorption wavelength of the blue photoelectric conversion film is not particularly limited, but is preferably in the range of 400 to 500 nm, and more preferably 420 to 480 nm.

As the conductive film and the transparent conductive film, the conductive film and the transparent conductive film included in the photoelectric conversion element according to the embodiment of the present invention can be used.

The configuration of the red photoelectric conversion element 24 is not particularly limited, but examples thereof include a photoelectric conversion element having a conductive film, a red photoelectric conversion film, and a transparent conductive film in this order.

The type of the red photoelectric conversion film is not particularly limited as long as it is a photoelectric conversion film capable of receiving red light, and includes an organic red photoelectric conversion film or an inorganic red photoelectric conversion film, and an organic red photoelectric conversion film (a red photoelectric conversion film composed of an organic compound) is preferable.

The maximum absorption wavelength of the red photoelectric conversion film is not particularly limited, but is preferably in the range of 600 to 700 nm, and more preferably 620 to 680 nm.

As the conductive film and the transparent conductive film, the conductive film and the transparent conductive film included in the photoelectric conversion element according to the embodiment of the present invention can be used.

In FIG. 3, the photoelectric conversion element according to the embodiment of the present invention, the blue photoelectric conversion element, and the red photoelectric conversion element are arranged in this order from the light incident side, but the arrangement is not limited to the aspect, and may be another aspect. For example, the blue photoelectric conversion element, the photoelectric conversion element according to the embodiment of the present invention, and the red photoelectric conversion element may be arranged in this order from the light incident side.

Among these, it is preferable that the photoelectric conversion element according to the embodiment of the present invention is arranged on the most light incident side. In this case, at least a part of the incidence ray is transmitted through the photoelectric conversion element according to the embodiment of the present invention, and then received by another photoelectric conversion element.

As described above, the configuration in which the photoelectric conversion elements of the three primary colors of blue, green, and red are laminated as the imaging element is described, but the configuration may be two layers (two colors) or four layers (four colors).

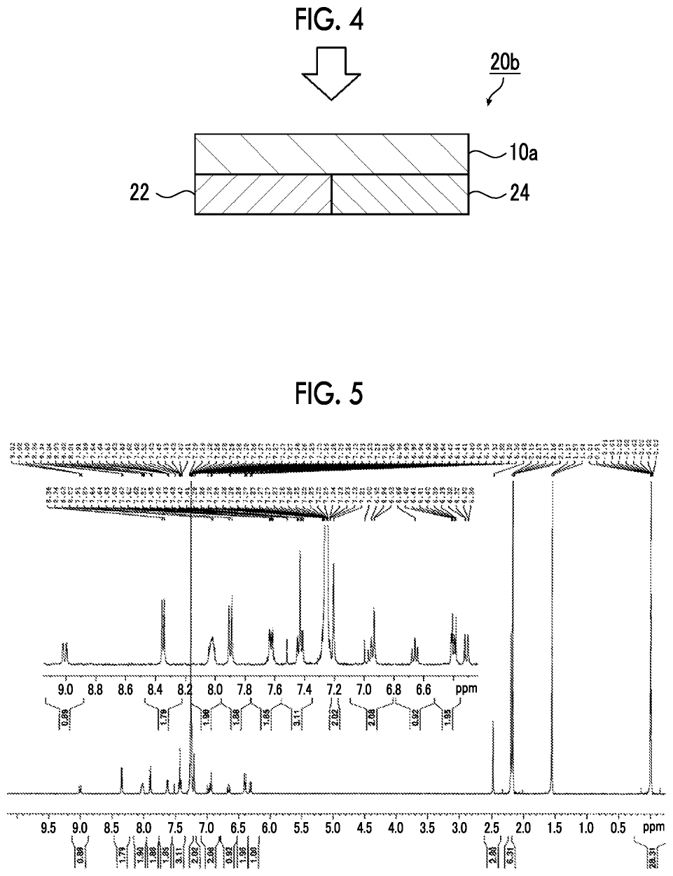

For example, as in the imaging element 20b shown in FIG. 4, the photoelectric conversion element 10a according to the embodiment of the present invention may be arranged on the arrayed blue photoelectric conversion element 22 and red photoelectric conversion element 24. In the case of the embodiment shown in FIG. 4, as needed, a color filter that absorbs light of a predetermined wavelength may be arranged on the light incident side.

The form of the imaging element is not limited to the forms shown in FIGS. 3 and 4, and may be other forms.

For example, the photoelectric conversion element according to the embodiment of the present invention, the blue photoelectric conversion element, and the red photoelectric conversion element may be arranged in the same plane position.

Examples of another application of the photoelectric conversion element include the photoelectric cell and the optical sensor, but the photoelectric conversion element according to the embodiment of the present invention is preferably used as the optical sensor. The photoelectric conversion element may be used alone as the optical sensor. Alternately, the photoelectric conversion element may be used as a line sensor in which the photoelectric conversion elements are linearly arranged or as a two-dimensional sensor in which the photoelectric conversion elements are planarly arranged.

### EXAMPLES

Hereinafter, the present invention will be described in more detail with reference to examples. The materials, usage amounts, proportion, processing contents, and processing procedures shown in the following examples can be appropriately changed without departing from the spirit of the present invention. Therefore, the scope of the present invention should not be construed as being limited by the following examples.

### Synthesis Example of Compound Represented by Formula (1)

Hereinafter, a synthesis method of the compound represented by Formula (1) will be described with a synthesis example of a compound (D-1) as an example.

<Synthesis of Compound (D-1)>

A compound (D-1) was synthesized according to the following scheme.

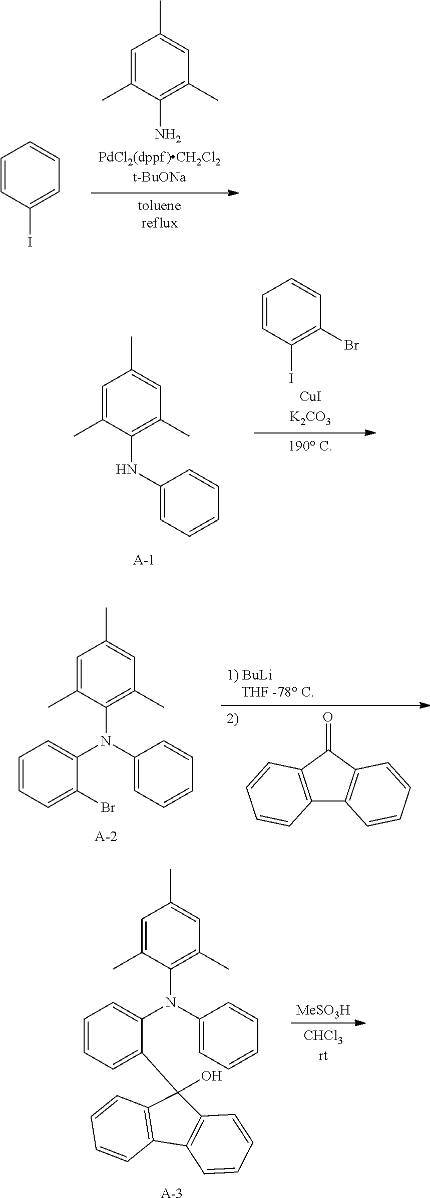

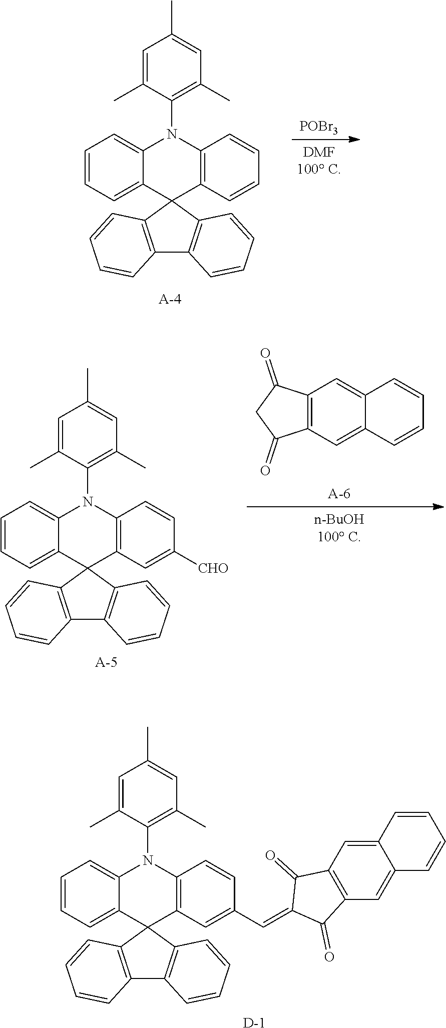

Iodobenzene (40.8 g, 200 mmol), 2,4,6-trimethylaniline (40.6 g, 300 mmol), and sodium t-butoxy sodium (28.8 g, 300 mmol) were added to toluene in the flask, and a series of operations of evacuation and nitrogen substitution in the flask was repeated three times in this order. [1,1′-Bis (diphenylphosphino) ferrocene] palladium (II) dichloride dichloromethane adduct (8.17 g, 10.0 mmol) was added to the obtained reaction solution, and the obtained reaction solution was reacted at 90° C. for 4 hours. The reaction solution was allowed to cool and then filtered, and the obtained filtrate was concentrated to obtain a crude product. The obtained crude product was purified by silica gel column chromatography (eluent: 10% ethyl acetate/hexane) to obtain a compound (A-1) (31.5 g, 150 mmol, yield 75%).

The compound (A-1) (26.0 g, 123 mmol), copper (I) iodide (8.93 g, 61.5 mmol), and tripotassium phosphate (52.2 g, 246 mmol) were added to 2-iodobromobenzene (104 g, 369 mmol) and the obtained reaction solution was reacted at 190° C. for 2 days. The reaction solution was allowed to cool and then filtered, and the obtained filtrate was concentrated to obtain a crude product. The obtained crude product was purified by silica gel column chromatography (eluent: 10% toluene/hexane) to obtain a compound (A-2) (27.6 g, 75.3 mmol, yield 61%).

The compound (A-2) (9.00 g, 24.6 mmol) was dissolved in tetrahydrofuran (125 mL), and the obtained solution was cooled to −78° C. Next, n-butyllithium (1.55M, 16.6 mL, 25.8 mmol) was added dropwise to the solution over 15 minutes. The obtained reaction solution was stirred at −78° C. for 30 minutes, and 9-fluorenone (4.88 g, 27.1 mmol) was further added. Next, the reaction solution was stirred at −78° C. for 30 minutes and then heated to 0° C., the reaction solution was added to an ammonium chloride aqueous solution, and ethyl acetate was added for extraction. The organic phase was collected, magnesium sulfate was added to the organic phase to dry the organic phase and filtered, and the obtained filtrate was concentrated to obtain a crude product (1). The obtained crude product (1) was dissolved in chloroform (250 mL), and methanesulfonic acid (2.36 g, 24.6 mmol) was added thereto. After stirring the obtained reaction solution at room temperature for 30 minutes, the reaction solution was added to an aqueous sodium hydrogen carbonate solution, and ethyl acetate was added for extraction. The organic phase was collected, magnesium sulfate was added to the organic phase to dry the organic phase and filtered, and the obtained filtrate was concentrated to obtain a crude product (2). The obtained crude product (2) was recrystallized from 2-propanol to obtain a compound (A-4) (9.60 g, 21.4 mmol, yield 86% (2 steps)).

The compound (A-4) (6.50 g, 14.5 mmol) was dissolved in N, N′-dimethylformamide (130 mL), and phosphorus oxybromide (16.6 g, 57.7 mmol) was added thereto. After reacting the obtained reaction solution at 90° C. for 6 hours, the reaction solution was allowed to cool and added to an aqueous sodium hydrogen carbonate solution, and ethyl acetate was added for extraction. The organic phase was collected, magnesium sulfate was added to the organic phase to dry the organic phase and filtered, and the obtained filtrate was concentrated to obtain a crude product. The obtained crude product was purified by silica gel column chromatography (eluent: 15% ethyl acetate/toluene) to obtain a compound (A-5) (4.62 g, 9.67 mmol, yield 67%).

The compound (A-5) (150 mg, 0.31 mmol) and a compound (A-6) (68 mg, 0.35 mmol) were added to n-butanol (1.0 mL) and reacted at 90° C. for 5 hours. The obtained reaction solution was allowed to cool, then methanol (3.0 mL) was added to the reaction solution and filtered to obtain a crude product. The obtained crude product was recrystallized from chlorobenzene/methanol (1:1, 6.0 mL) to obtain the compound (D-1) (180 mg, 0.27 mmol, yield 87%).

The obtained compound (D-1) was identified by NMR and mass spectrometry (MS).

¹H NMR spectrum (400 MHz, CDCl₃) is shown in FIG. 5.

¹MS(ESI⁺) m/z: 656.3 ([M+H]⁺)

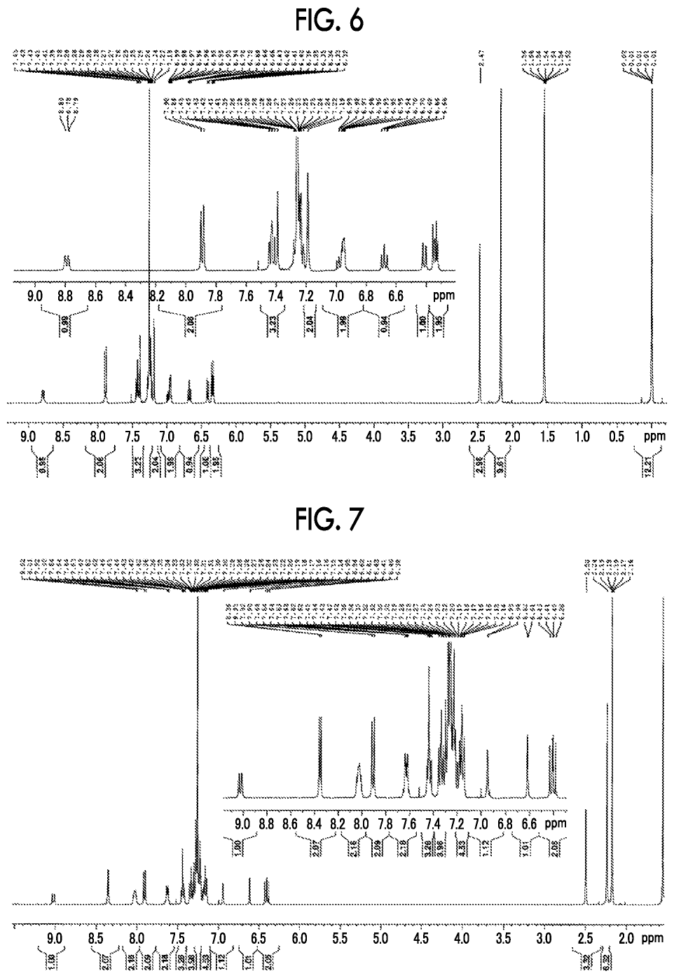

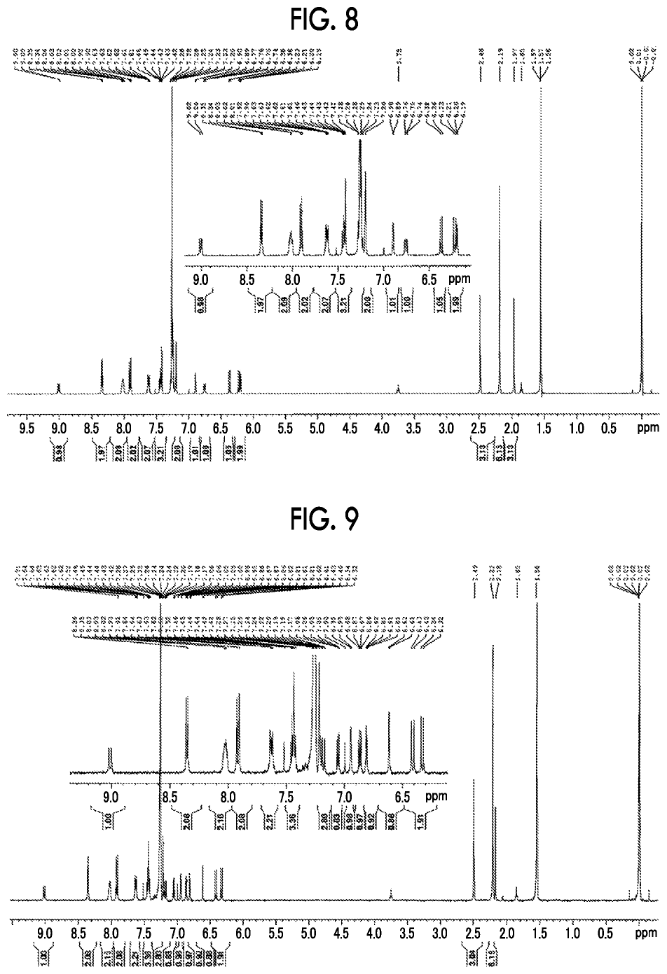

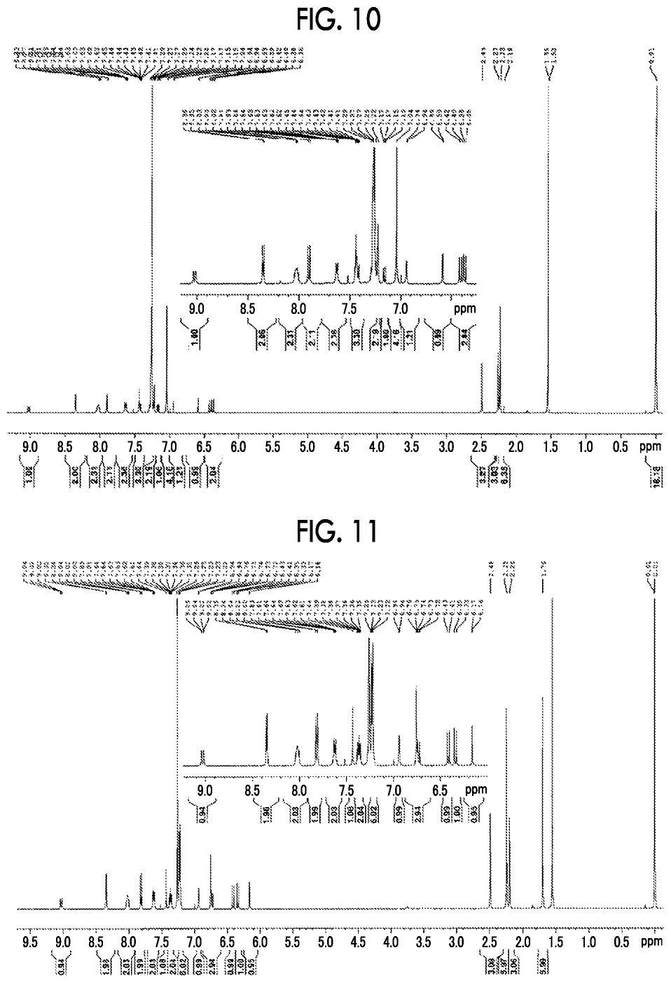

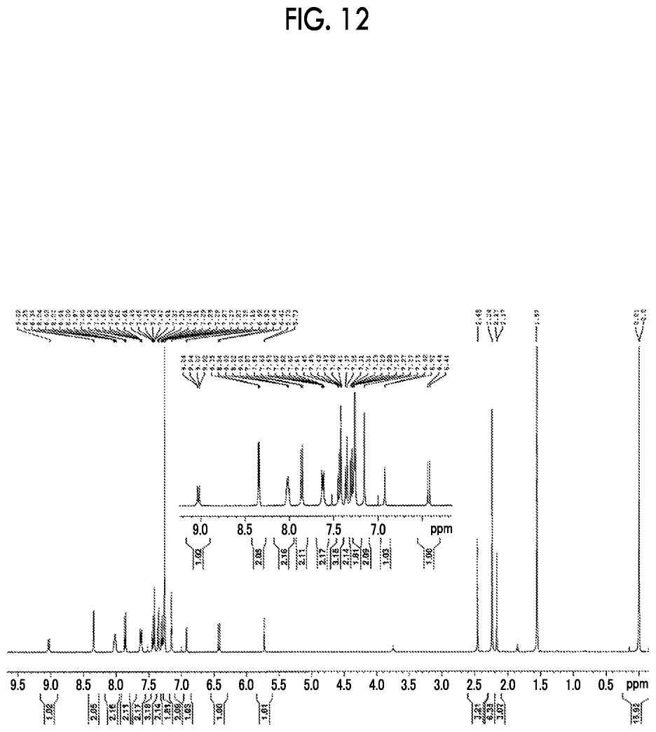

With reference to the synthesis method of the compound (D-1), compounds (D-2) to (D-12) shown in Example were synthesized. ¹H NMR spectra (400 MHz, CDCl₃) of the compounds (D-2), (D-6), and (D-11) are shown in FIGS. 6 to 12.

The structures of the compounds (D-1) to (D-12) and the comparative compounds (R-1) to (R-2) are shown below.

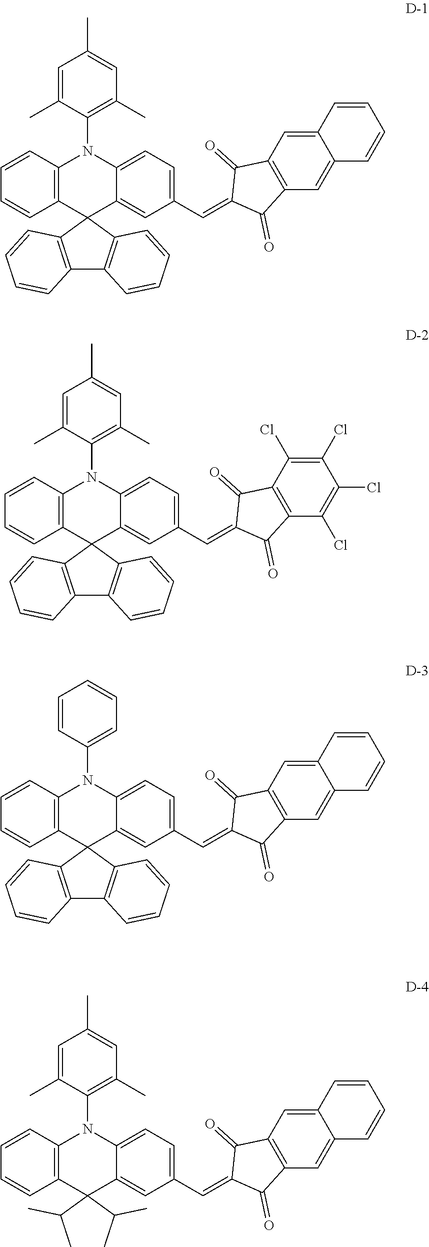 [SMILES: Cc1cc(C)c(N2c3ccccc3C3(c4ccccc4-c4ccccc43)c3cc(C=C4C(=O)c5cc6ccccc6cc5C4=O)ccc32)c(C)c1 ; Cc1cc(C)c(N2c3ccccc3C3(c4ccccc4-c4ccccc43)c3cc(C=C4C(=O)c5c(Cl)c(Cl)c(Cl)c(Cl)c5C4=O)ccc32)c(C)c1 ; O=C1C(=Cc2ccc3c(c2)C2(c4ccccc4-c4ccccc42)c2ccccc2N3c2ccccc2)C(=O)c2cc3ccccc3cc21 ; Cc1cc(C)c(N2c3ccccc3C(C(C)C)(C(C)C)c3cc(C=C4C(=O)c5cc6ccccc6cc5C4=O)ccc32)c(C)c1]

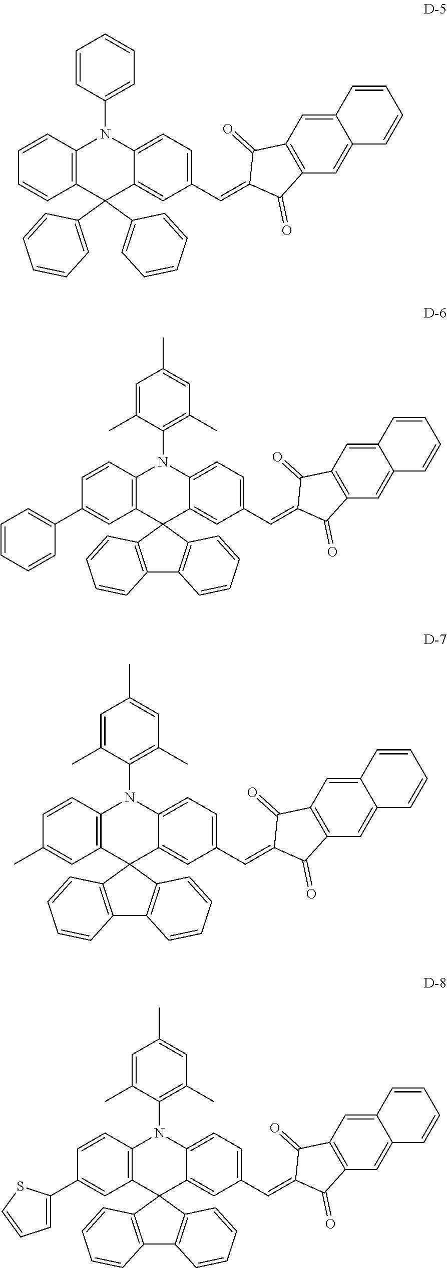 [SMILES: O=C1C(=Cc2ccc3c(c2)C(c2ccccc2)(c2ccccc2)c2ccccc2N3c2ccccc2)C(=O)c2cc3ccccc3cc21 ; Cc1cc(C)c(N2c3ccc(C=C4C(=O)c5cc6ccccc6cc5C4=O)cc3C3(c4ccccc4-c4ccccc43)c3cc(-c4ccccc4)ccc32)c(C)c1 ; Cc1cc(C)c(N2c3ccc(C)cc3C3(c4ccccc4-c4ccccc43)c3cc(C=C4C(=O)c5cc6ccccc6cc5C4=O)ccc32)c(C)c1 ; Cc1cc(C)c(N2c3ccc(C=C4C(=O)c5cc6ccccc6cc5C4=O)cc3C3(c4ccccc4-c4ccccc43)c3cc(-c4cccs4)ccc32)c(C)c1]

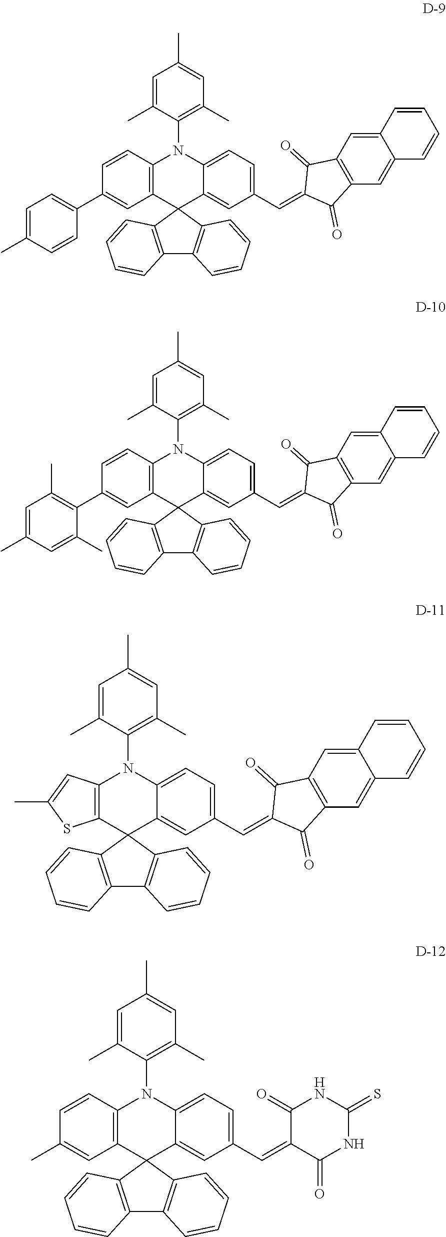 [SMILES: Cc1ccc(-c2ccc3c(c2)C2(c4ccccc4-c4ccccc42)c2cc(C=C4C(=O)c5cc6ccccc6cc5C4=O)ccc2N3c2c(C)cc(C)cc2C)cc1 ; Cc1cc(C)c(-c2ccc3c(c2)C2(c4ccccc4-c4ccccc42)c2cc(C=C4C(=O)c5cc6ccccc6cc5C4=O)ccc2N3c2c(C)cc(C)cc2C)c(C)c1 ; Cc1cc(C)c(N2c3ccc(C=C4C(=O)c5cc6ccccc6cc5C4=O)cc3C3(c4ccccc4-c4ccccc43)c3sc(C)cc32)c(C)c1 ; Cc1cc(C)c(N2c3ccc(C)cc3C3(c4ccccc4-c4ccccc43)c3cc(C=C4C(=O)NC(=S)NC4=O)ccc32)c(C)c1]

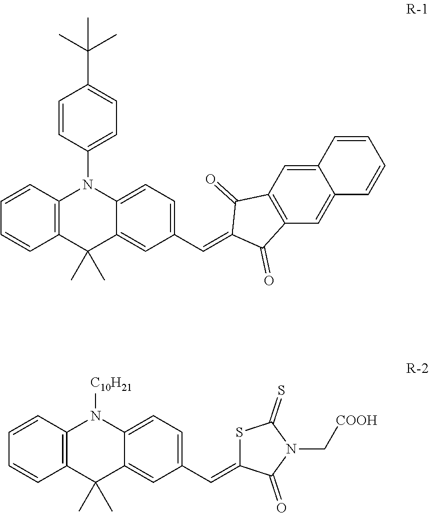

<Production of Deposited Film>

The compounds (D-1 to D-12, and R-1 to R-2) obtained by the vacuum evaporation method were vapor-deposited to form films under the condition that the temperature of the glass substrate is controlled to 25° C., and vapor deposited films having the thickness of 100 nm was formed on the glass substrate.

<Measurement of Absorption Waveform of Deposited Film>

The absorption shape of the obtained vapor deposited film was measured using a spectrophotometer U3310 manufactured by Hitachi High-Tech Co., Ltd. Table 1 shows the maximum absorption wavelength of the obtained absorption spectrum and the width of the light absorbance of 0.5 (the absorption half-width) in a case where the light absorbance of the maximum absorption wavelength is normalized to 1.

In Table 1, the column of “corresponding to Formula (3)” is “A” in a case where each compound corresponds to the compound represented by Formula (3), and “B” in the opposite case.

In Table 1, the column of “corresponding to Formula (4)” is “A” when each compound corresponds to the compound represented by Formula (4), and “B” in the opposite case.

TABLE 1

| | | | Maximum | |
|---|---|---|---|---|
| | Correspond- | Correspond- | absorption | Absorption |
| Compound | ing to | ing to | wavelength | half-width |
| Type | Formula (3) | Formula (4) | (nm) | (nm) |
| D-1 | A | A | 526 | 79 |
| D-2 | A | A | 541 | 80 |
| D-3 | A | B | 519 | 89 |
| D-4 | A | A | 540 | 85 |
| D-5 | A | B | 510 | 89 |
| D-6 | A | A | 540 | 85 |
| D-7 | A | A | 536 | 80 |
| D-8 | A | A | 545 | 85 |
| D-9 | A | A | 543 | 85 |
| D-10 | A | A | 539 | 79 |
| D-11 | B | B | 524 | 93 |
| D-12 | B | B | 544 | 94 |
| R-1 | — | — | 528 | 102 |

| | | | |
|---|---|---|---|
| R-2 | — | — | Unable to form film |

As shown in Table 1, the half-width of the absorption peak was narrow in the vapor deposited film of the specific compound.

A vapor deposited film could not be obtained in a case where the compound (R-2) was used.

### Examples and Comparative Examples: Production of Photoelectric Conversion Element

The photoelectric conversion element of the form of FIG. 1 was produced using the obtained compound. Here, the photoelectric conversion element includes a lower electrode 11, an electron blocking film 16A, a photoelectric conversion film 12, and an upper electrode 15.

Specifically, an amorphous ITO was formed into a film on the glass substrate by the sputtering method to form the lower electrode 11 (a thickness: 30 nm). Furthermore, the compound (EB-1) was formed into a film on the lower electrode 11 by the vacuum heating evaporation method to form the electron blocking film 16A (a thickness: 30 nm).

Furthermore, the compound (D-1) and the fullerene (C₆₀) were subjected to co-vapor deposition by the vacuum evaporation method so as to be respectively 100 μm and 50 nm in terms of single layer on the electron blocking film 16A to form a film in a state where the temperature of the substrate was controlled to 25° C., and the photoelectric conversion film 12 having the bulk hetero structure of 150 nm was formed.

Furthermore, amorphous ITO was formed into a film on the photoelectric conversion film 12 by a sputtering method to form the upper electrode 15 (the transparent conductive film) (the thickness: 10 nm). After the SiO film was formed as the sealing layer on the upper electrode 15 by a vacuum evaporation method, an aluminum oxide (Al₂O₃) layer was formed thereon by an atomic layer chemical vapor deposition (ALCVD) method to produce a photoelectric conversion element.

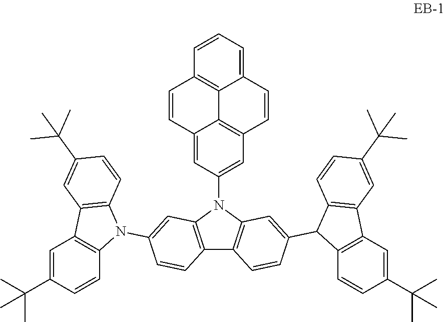 [SMILES: CC(C)(C)c1ccc2c(c1)-c1cc(C(C)(C)C)ccc1C2c1ccc2c3ccc(-n4c5ccc(C(C)(C)C)cc5c5cc(C(C)(C)C)ccc54)cc3n(-c3cc4ccc5cccc6ccc(c3)c4c56)c2c1]

Similarly, the photoelectric conversion elements were produced using the compounds (D-2) to (D-12) and (R-1).

As described above, with the compound (R-2), a vapor deposited film was not in the first place, and a photoelectric conversion element could not be produced.

<Confirmation of Driving (Evaluation of Photoelectric Conversion Efficiency (External Quantum Efficiency))>

The driving of each of the obtained photoelectric conversion elements was confirmed. A voltage was applied to each photoelectric conversion element so that the electric field strength was 2.0×10⁵ V/cm. Then, the photoelectric conversion efficiency (the external quantum efficiency) at 540 nm was measured by irradiating light from the upper electrode (the transparent conductive film) side, and it was confirmed that all of the photoelectric conversion elements manufactured by using the compounds (D-1) to (D-10) and (R-1) exhibit a photoelectric conversion efficiency of 60% or more, and have sufficient external quantum efficiency as a photoelectric conversion element. The external quantum efficiency was measured using a constant energy quantum efficiency measuring device manufactured by Optel. The irradiation light amount was 50 μW/cm².

<Heat Resistance Evaluation>

The heat resistance of each obtained photoelectric conversion element was evaluated. Specifically, each obtained photoelectric conversion element was heated on a hot plate at 180° C. for 30 minutes. A voltage was applied to each photoelectric conversion element after heating so that the electric field strength was 2.0×10⁵ V/cm, and was measured by irradiating light from the upper electrode (the transparent conductive film) side to photoelectric conversion efficiency at 540 nm (the external quantum efficiency). The external quantum efficiency was measured using a constant energy quantum efficiency measuring device manufactured by Optel. The evaluation was performed by the relative value of the photoelectric conversion efficiency after heating in a case where the photoelectric conversion efficiency before heating is 1. The evaluation was performed with a relative value of 0.90 or more being evaluated as A, a relative value of 0.80 or more and less than 0.90 being B, and a relative value of less than 0.80 being C. In practice, B or more is preferable, and A is more preferable.

The absorption half-width of the photoelectric conversion film in the obtained photoelectric conversion element was evaluated according to the same procedure as described above in <Measurement of Absorption Waveform of Deposited Film>, and the results are shown in Table 2.

In Table 2, the notation methods of the “corresponding to Formula (3)” column and the “corresponding to Formula (4)” column are the same as those in Table 1 described above.

TABLE 2

| | | Correspond- | Correspond- | Absorption | Heat |
|---|---|---|---|---|---|
| | | ing to | ing to | half-width | resis- |
| | Type | Formula (3) | Formula (4) | (nm) | tance |
| Example 1 | D-1 | A | A | 79 | A |
| Example 2 | D-2 | A | A | 81 | A |
| Example 3 | D-3 | A | B | 89 | A |
| Example 4 | D-4 | A | A | 86 | A |
| Example 5 | D-5 | A | B | 88 | A |
| Example 6 | D-6 | A | A | 87 | A |
| Example 7 | D-7 | A | A | 82 | A |
| Example 8 | D-8 | A | A | 84 | A |
| Example 9 | D-9 | A | A | 85 | A |
| Example 10 | D-10 | A | A | 79 | A |
| Example 11 | D-11 | B | B | 95 | A |
| Example 12 | D-12 | B | B | 94 | B |
| Comparative | R-1 | — | — | 101 | C |
| Example 1 | | | | | |

As shown in Table 2, it was confirmed that the photoelectric conversion element according to the embodiment of the present invention has a narrower absorption half-width and higher heat resistance than the comparative example.

Among these, as shown in Examples above, in a case where the compound represented by Formula (3) is used, the absorption half-width becomes narrower, and in a case where the compound represented by Formula (4) is used, the absorption half-width becomes further narrower.

<Production of Imaging Element>

The same imaging elements as shown in FIG. 3 were produced using the compounds (D-1) to (D-12).

The photoelectric conversion element functioning as a green photoelectric conversion element was produced by the method described above.

The blue photoelectric conversion element and the red photoelectric conversion element were produced with reference to the description of JP2005-303266A.

In the obtained imaging element, since the absorption peak of the photoelectric conversion film in the photoelectric conversion element according to the embodiment of the present invention has a narrow half-width, light was easily received by the blue photoelectric conversion element and the red photoelectric conversion element, and color separation performance was excellent.

### EXPLANATION OF REFERENCES

10a, 10b: photoelectric conversion element
11: conductive film (lower electrode)
12: photoelectric conversion film
15: transparent conductive film (upper electrode)
16A: electron blocking film
16B: positive hole blocking film
20a, 20b: imaging element
22: blue photoelectric conversion element
24: red photoelectric conversion element

What is claimed is:

1. A photoelectric conversion element comprising:
a conductive film;
a photoelectric conversion film; and
a transparent conductive film, in this order,
wherein the photoelectric conversion film contains a compound represented by Formula (1), 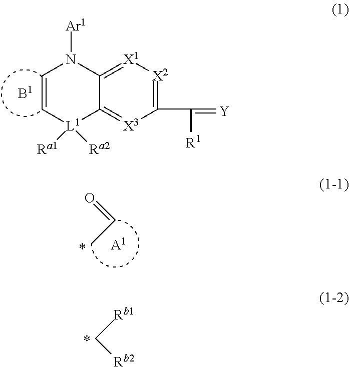
in Formula (1), Ar¹ represents an aryl group which may have a substituent or a heteroaryl group which may have a substituent, R¹ represents a hydrogen atom or a substituent, X¹ to X³ each independently represent CR² or a nitrogen atom, R² represents a hydrogen atom or a substituent, R^{a1} and R^{a2} each independently represent a substituent having 2 or more carbon atoms, R^{a1} and R^{a2} may bond to each other to form a ring, L¹ represents a carbon atom, a silicon atom, or a germanium atom, B represents an aromatic ring which may have a substituent, Y represents a group represented by Formula (1-1) or a group represented by Formula (1-2), A¹ represents a ring containing at least two carbon atoms, R^{b1} and R^{b2} each independently represent a cyano group or —COOR^{d1}, R^{d1} represents an alkyl group or an aryl group, and * represents a bonding position,
where, the compound represented by Formula (1) has none of a carboxy group, a salt of a carboxy group, a phosphoric acid group, a salt of a phosphoric acid group, a sulfonic acid group, or a salt of a sulfonic acid group.

2. The photoelectric conversion element according to claim 1, wherein the compound represented by Formula (1) is a compound represented by Formula (2), 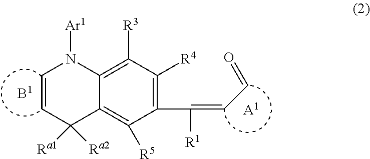
in Formula (2), Ar¹ represents an aryl group which may have a substituent or a heteroaryl group which may have a substituent, R¹ and R³ to R⁵ each independently represent a hydrogen atom or a substituent, R^{a1} and R^{a2} each independently represent a substituent having 2 or more carbon atoms, R^{a1} and R^{a2} may bond to each other to form a ring, B¹ represents an aromatic ring which may have a substituent, and A¹ represents a ring containing at least two carbon atoms,
where, the compound represented by Formula (2) has none of a carboxy group, a salt of a carboxy group, a phosphoric acid group, a salt of a phosphoric acid group, a sulfonic acid group, or a salt of a sulfonic acid group.

3. The photoelectric conversion element according to claim 1, wherein the compound represented by Formula (1) is a compound represented by Formula (3), 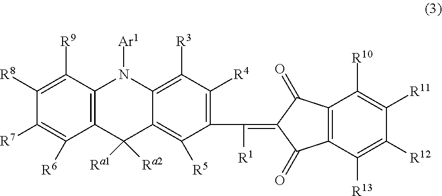
in Formula (3), Ar¹ represents an aryl group which may have a substituent or a heteroaryl group which may have a substituent, R¹ and R³ to R¹³ each independently represent a hydrogen atom or a substituent, R¹⁰ and R¹¹, R¹¹ and R¹², and R¹² and R¹³ may respectively independently bond to each other to form a ring, R^{a1} and R^{a2} each independently represent a substituent having 2 or more carbon atoms, and R^{a1} and R^{a2} may bond to each other to form a ring,
where, the compound represented by Formula (3) has none of a carboxy group, a salt of a carboxy group, a phosphoric acid group, a salt of a phosphoric acid group, a sulfonic acid group, or a salt of a sulfonic acid group.

4. The photoelectric conversion element according to claim 1, wherein the compound represented by Formula (1) is a compound represented by Formula (4), 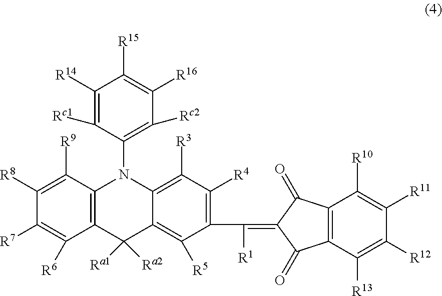
in Formula (4), R¹ and R³ to R¹⁶ each independently represent a hydrogen atom or a substituent, R¹⁰ and R¹¹, R¹¹ and R¹², and R¹² and R¹³ may respectively independently bond to each other to form a ring, R^{a1} and R^{a2} each independently represent a substituent having 2 or more carbon atoms, R^{a1} and R^{a2} may bond to each other to form a ring, and R^{c1} and R^{c2} each independently represent a hydrogen atom or a substituent, and at least one of R^{c1} or R^{c2} represents a substituent,
where, the compound represented by Formula (4) has none of a carboxy group, a salt of a carboxy group, a phosphoric acid group, a salt of a phosphoric acid group, a sulfonic acid group, or a salt of a sulfonic acid group.

5. The photoelectric conversion element according to claim 1, wherein R^{a1} and R^{a2} each independently represent a substituent having 3 or more carbon atoms.

6. The photoelectric conversion element according to claim 1, wherein R^{a1} and R^{a2} each independently represent a secondary alkyl group having 3 or more carbon atoms, an aryl group having 3 or more carbon atoms, or a heteroaryl group having 3 or more carbon atoms.

7. The photoelectric conversion element according to claim 1, wherein the photoelectric conversion film further contains an n-type organic semiconductor, and has a bulk hetero structure formed in a state where the compound represented by Formula (1) and the n-type organic semiconductor are mixed.

8. The photoelectric conversion element according to claim 1, further comprising one or more interlayers between the conductive film and the transparent conductive film, in addition to the photoelectric conversion film.

9. An imaging element comprising the photoelectric conversion element according to claim 1.

10. The imaging element according to claim 9, further comprising another photoelectric conversion element that receives light having a wavelength different from light received by the photoelectric conversion element.

11. The imaging element according to claim 10,
wherein the photoelectric conversion element and the other photoelectric conversion element are laminated, and
at least a part of incidence ray is transmitted through the photoelectric conversion element, and then is received by the other photoelectric conversion element.

12. The imaging element according to claim 10,
wherein the photoelectric conversion element is a green photoelectric conversion element, and
the other photoelectric conversion element includes a blue photoelectric conversion element and a red photoelectric conversion element.

13. An optical sensor comprising the photoelectric conversion element according to claim 1.

14. A compound represented by Formula (1), 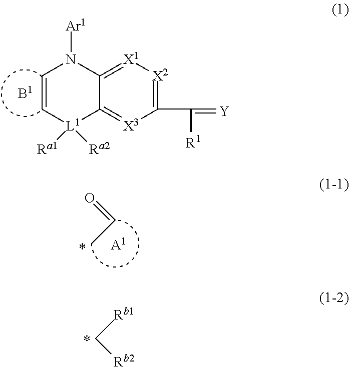
in Formula (1), Ar¹ represents an aryl group which may have a substituent or a heteroaryl group which may have a substituent, R¹ represents a hydrogen atom or a substituent, X¹ to X³ each independently represent CR² or a nitrogen atom, R² represents a hydrogen atom or a substituent, R^{a1} and R^{a2} each independently represent a substituent having 2 or more carbon atoms, R^{a1} and R^{a2} may bond to each other to form a ring, L¹ represents a carbon atom, a silicon atom, or a germanium atom, B¹ represents an aromatic ring which may have a substituent, Y represents a group represented by Formula (1-1) or a group represented by Formula (1-2), A¹ represents a ring containing at least two carbon atoms, R^{b1} and R^{b2} each independently represent a cyano group or —COOR^{d1}, R^{d1} represents an alkyl group or an aryl group, and * represents a bonding position,
where, the compound represented by Formula (1) has none of a carboxy group, a salt of a carboxy group, a phosphoric acid group, a salt of a phosphoric acid group, a sulfonic acid group, or a salt of a sulfonic acid group.

15. The compound according to claim 14 which is represented by Formula (2), 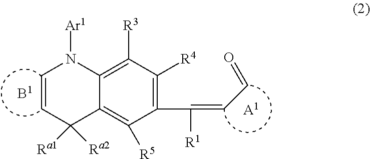
in Formula (2), Ar¹ represents an aryl group which may have a substituent or a heteroaryl group which may have a substituent, R¹ and R³ to R⁵ each independently represent a hydrogen atom or a substituent, R^{a1} and R^{a2} each independently represent a substituent having 2 or more carbon atoms, R^{a1} and R^{a2} may bond to each other to form a ring, B¹ represents an aromatic ring which may have a substituent, and A¹ represents a ring containing at least two carbon atoms,
where, the compound represented by Formula (2) has none of a carboxy group, a salt of a carboxy group, a phosphoric acid group, a salt of a phosphoric acid group, a sulfonic acid group, or a salt of a sulfonic acid group.

16. The compound according to claim 14 which is represented by Formula (3), 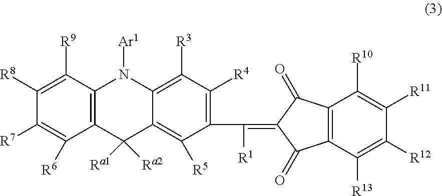
in Formula (3), Ar¹ represents an aryl group which may have a substituent or a heteroaryl group which may have a substituent, R¹ and R³ to R¹³ each independently represent a hydrogen atom or a substituent, R¹⁰ and R¹¹, R¹¹ and R¹², and R¹² and R¹³ may respectively independently bond to each other to form a ring, R^{a1} and R^{a2} each independently represent a substituent having 2 or more carbon atoms, and R^{a1} and R^{a2} may bond to each other to form a ring,
where, the compound represented by Formula (3) has none of a carboxy group, a salt of a carboxy group, a phosphoric acid group, a salt of a phosphoric acid group, a sulfonic acid group, or a salt of a sulfonic acid group.

17. The compound according to claim 14 which is represented by Formula (4), 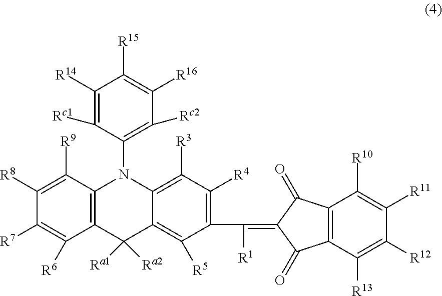
in Formula (4), R¹ and R³ to R¹⁶ each independently represent a hydrogen atom or a substituent, R¹⁰ and R¹¹, R¹¹ and R¹², and R¹² and R¹³ may respectively independently bond to each other to form a ring, R^{a1} and R^{a2} each independently represent a substituent having 2 or more carbon atoms, R^{a1} and R^{a2} may bond to each other to form a ring, and R^{c1} and R^{c2} each independently represent a hydrogen atom or a substituent, and at least one of R^{c1} or R^{c2} represents a substituent,
where, the compound represented by Formula (4) has none of a carboxy group, a salt of a carboxy group, a phosphoric acid group, a salt of a phosphoric acid group, a sulfonic acid group, or a salt of a sulfonic acid group.

18. The compound according to claim 14, wherein R^{a1} and R^{a2} each independently represent a substituent having 3 or more carbon atoms.

19. The compound according to claim 14, wherein R^{a1} and R^{a2} each independently represent a secondary alkyl group having 3 or more carbon atoms, an aryl group having 3 or more carbon atoms, or a heteroaryl group having 3 or more carbon atoms.

20. The photoelectric conversion element according to claim 2, wherein the compound represented by Formula (1) is a compound represented by Formula (3), 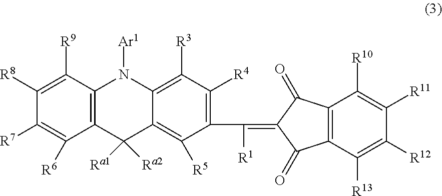
in Formula (3), Ar¹ represents an aryl group which may have a substituent or a heteroaryl group which may have a substituent, R¹ and R³ to R¹³ each independently represent a hydrogen atom or a substituent, R¹⁰ and R¹¹, R¹¹ and R¹², and R¹² and R¹³ may respectively independently bond to each other to form a ring, R^{a1} and R^{a2} each independently represent a substituent having 2 or more carbon atoms, and R^{a1} and R^{a2} may bond to each other to form a ring,
where, the compound represented by Formula (3) has none of a carboxy group, a salt of a carboxy group, a phosphoric acid group, a salt of a phosphoric acid group, a sulfonic acid group, or a salt of a sulfonic acid group.
